(19) United States
(12) Patent Application Publication
(10) Pub. No.: US 2007/0062525 A1
(43) Pub. Date: Mar. 22, 2007

(54) Medicament dispenser

(76) Inventors: Stanley George Bonney, Ware (GB); Michael Birsha Davies, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/502,405

(22) PCT Filed: Jan. 22, 2003
(86) PCT No.: PCT/EP03/00599

§ 371 Date: Feb. 7, 2005

(30) Foreign Application Priority Data
Jan. 25, 2002 (GB) 0201677.2
Jul. 25, 2002 (GB) 0217199.9

(51) Int. Cl. A61M 15/00; B65D 83/06

(52) U.S. Cl. 128/203.21; 128/203.15

(57) ABSTRACT

There is provided a medicament dispenser for use in the delivery of a combination medicament product. The dispenser comprises first and second medicament containers for containing first and second medicament active component; and first and second release means for releasing respective dose portions of the first and second medicament active component from the first and second medicament containers. The first medicament active component is kept separate from the second medicament active component until the point of release thereof for delivery in combination. The first medicament active component is selected from the group consisting of salmeterol, formoterol and any salts or solvates thereof. The second medicament active component is selected from the group consisting of beclomethasone ester, fluticasone ester and any salts or solvates thereof.

### TECHNICAL FIELD

The present invention relates to a medicament dispenser for dispensing medicament. The invention particularly relates to a dispenser for use in dispensing a combination medicament product in powder or aerosol form.

### BACKGROUND TO THE INVENTION

The use of inhalation devices in the administration of medicaments, for example in bronchodilation therapy is well known. Such devices generally comprise a body or housing within which a medicament carrier is located. Known inhalation devices include dry powder inhaler (DPI) devices, including those in which the medicament carrier is a blister pack containing a number of discrete doses of powdered medicament. Such devices usually contain a mechanism of accessing these doses, usually comprising either blister piercing means or peeling means. The powdered medicament can then be accessed and inhaled. Other known devices include reservoir dry powder inhalers and those in which the medicament is delivered in aerosol form, including the well-known metered dose inhaler (MDI) delivery devices.

Therapies involving combinations of different and complementary active medicaments are known. These can be administered either as distinct combination (i.e. multi-active) medicament products, which comprise a defined mixture of each component medicament, or as groups of single active medicament products, which are designed to be taken in combination or sequentially. Whilst combination products offer added convenience for the patient, certain medicament actives are difficult to formulate as distinct combination products. For example, the actives may interact chemically with each other in an undesirable way when formulated together.

It is thus, desirable in certain circumstances, to have a medicament dispenser that separately (i.e. in isolated fashion) contains each active component (or mixture thereof) of a combination product, but which enables the delivery of a combined dose in response to a minimum number of patient actions. In particular, it is desirable that all active components of the combined dose are delivered to the patient in a single, combined dose in response to a single patient dosing action. For example, it is desirable that a combination inhaled medicament product be delivered in response to a single actuation of an inhaler, even where the active components of that combined product are separately stored within the inhaler device.

The Applicants have now devised a combination medicament dispenser device arranged to accommodate separately located active components as defined herein. In one aspect, the invention provides the ability to tailor the composition of the multi-active component ‘combined product’ by varying the relative ratio of release of its active medicament component parts and therefore to enable greater flexibility of dosing of combination product to the patient.

### SUMMARY OF THE INVENTION

According to one aspect of the present invention there is provided a medicament dispenser for use in the delivery of a combination medicament product, the dispenser comprising

a first medicament container for containing a first medicament active component;

a first release means for releasing a dose portion of said first medicament active component from said first medicament container;

a second medicament container for containing a second medicament active component; and

a second release means for releasing a dose portion of said first medicament active component from said second medicament container,

wherein the first medicament active component is kept separate from the second medicament active component until the point of release thereof for delivery in combination.

The first medicament active component is selected from the group consisting of salmeterol, formoterol and any salts or solvates thereof. Preferably, the first medicament active component is selected from the group consisting of salmeterol xinafoate and formoterol fumarate.

The second medicament active component is selected from the group consisting of beclomethasone ester, fluticasone ester and any salts or solvates thereof. Preferably, the second medicament active component is selected from the group consisting of beclomethasone dipropionate and a fluticasone propionate.

In a first particularly preferred combination, the first medicament active component is salmeterol xinafoate and the second medicament active component is fluticasone propionate.

In a second particularly preferred combination, the first medicament active component is formoterol fumarate and the second medicament active component is fluticasone propionate.

In a third particularly preferred combination, the first medicament active component is formoterol fumarate and the second medicament active component is beclomethasone dipropionate.

In combination, the dose portions of said first and second medicament active component releasable from the first and second medicament containers comprise a defined dose of combination product. That is to say, that when combined together (e.g. on release) the distinct active medicament dose portions form a single dose of a ‘multi-active’ medicament treatment.

In one particular aspect, the first and second medicament active components are co-formulation incompatible. The term ‘co-formulation incompatible’ herein is used to mean incompatible in the sense of not being amenable to co-formulation for whatever reason including chemical or physical incompatibility or complexity of formulation development. That is to say, for whatever reason, including development simplicity, the medicament active components are preferably not co-formulated.

In one particular aspect, the dispenser device is designed to receive the first and second medicament container only (i.e. two medicament containers only).

Suitably, the first and second medicament containers are of a similar-type. Their sizing may be identical, or in other aspects the size of each container may differ to e.g. reflect differences in dosing volume of each medicament active.

In one aspect, the first and second medicament container are of a type adapted to be used with a medicament dispenser selected from the group consisting of a unit dose dry powder inhaler (UDPI), a reservoir dry powder inhaler (RDPI), a multi-dose dry powder inhaler (MDPI), and a metered dose inhaler (MDI).

By unit dose dry powder inhaler (UDPI) is meant an inhaler suitable for dispensing medicament in dry powder form, wherein each medicament active component is comprised within a unit dose container pack containing (or otherwise carrying) a single define dose portion of medicament active product. In a preferred aspect, the carrier has a capsule form, but it could also, for example, comprise a blister pack form or a carrier onto which medicament has been applied by any suitable process including printing, painting and vacuum occlusion.

By reservoir dry powder inhaler (RDPI) it is meant an inhaler having a reservoir form container pack suitable for containing multiple (un-metered doses) of medicament product in dry powder form and including means for metering a medicament dose portion from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose container pack containing (or otherwise carrying) multiple, defines dose portions of active medicament product. In a preferred aspect, the carrier has a blister pack form, but it could also, for example, comprise a multiple capsule-based pack form or a carrier onto which medicament has been applied by any suitable process including printing, painting and vacuum occlusion.

In one aspect, the multi-dose pack is a blister pack comprising multiple blisters for containment of medicament product in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 in the name of Glaxo Group Ltd describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

In one aspect herein, the medicament dispenser is of the MDPI type and designed for use with plural elongate form dry powder medicament carriers, each having multiple distinct medicament dose portions carried thereby, said dispenser having a dispensing mechanism for dispensing the distinct medicament dose portions carried by each of said plural medicament carriers, said mechanism comprising,

a) a receiving station for receiving each of the plural medicament carriers;

b) a release for releasing a distinct medicament dose portion from each of the plural medicament carriers on receipt thereof by said receiving station;

c) an outlet, positioned to be in communication with the distinct medicament dose portions releasable by said release; and

d) an indexer for individually indexing the distinct medicament dose portions of each of the plural medicament carriers.

The medicament dispenser is designed to receive plural elongate form medicament carriers. Preferably, the medicament dispenser is designed to receive two such carriers.

Each elongate form medicament carrier has multiple distinct dose portions carried thereby. The distinct dose portions are typically arranged in spaced fashion, more preferably in progressive arrangement (e.g. series progression) on the carrier such that each dose portion is separately accessible.

The term elongate medicament carrier herein is used to define any suitable form of elongate carrier for medicament. Suitably, each elongate form medicament carrier is in the form of a strip or tape. In one preferred aspect, the carrier has a blister pack form, but it could also, for example, comprise a carrier onto which medicament has been applied by any suitable process including printing, painting and vacuum occlusion.

In one aspect, the elongate form medicament carrier comprises a blister pack in laminate form. Suitably, the laminate comprises material selected from the group consisting of metal foil, organic polymeric material and paper. Suitable metal foils include aluminium or tin foil having a thickness of from 5 to 100 μm, preferably from 10 to 50 μm, such as 20 to 30 μm. Suitable organic polymeric materials include polyethylene, polypropylene, polyvinyl chloride and polyethylene terephthalate.

Access to the medicament dose portions comprised within the pockets of the elongate strip form carrier is by any suitable access means including tearing, piercing or peeling apart the relevant pockets.

One suitable blister pack form medicament carrier comprises a peelable blister strip. Suitably, the peelable blister strip comprises a base sheet in which blisters are formed to define pockets therein for containing distinct medicament dose portions and a lid sheet which is hermetically sealed to the base sheet except in the region of the blisters in such a manner that the lid sheet and the base sheet can be peeled apart. The base and lid sheets are typically sealed to one another over their whole width except for the forward end portions where they are typically not sealed to one another at all. Thus, separate base and lid sheet forward end portions are presented at the end of the strip. The respective base and lid sheets are peelably separable from each other to (e.g. separately) release the contents of each pocket.

Suitably, the lid sheet comprises at least the following successive layers: (a) paper; adhesively bonded to (b) polyester; adhesively bonded to (c) aluminium foil; that is coated with a heat seal lacquer for bonding to the base sheet. The thickness of each layer may be selected according to the desired properties but is typically of the order of from 5 to 200 micron, particularly from 10 to 50 micron.

Suitably, the base sheet comprises at least the following successive layers: (a) oriented polyamide (OPA); adhesively bonded to (b) aluminium foil; adhesively bonded to (c) a third layer comprising a polymeric material (e.g. polyvinyl chloride).

Various known techniques can be employed to join the lid and base sheet and hence to seal the blisters of the peelable blister strip. Such methods include adhesive bonding, hot metal bonding, hot metal welding, radio frequency welding, laser welding, ultrasonic welding and hot bar sealing. The lid sheet and base sheet of the peelable blister strip are particularly sealable by ‘cold form’ sealing methods, which are conducted at lower temperatures than conventional heat sealing methods. Such ‘cold form’ sealing methods are of particular utility where the medicament or medicament formulation for containment within the blister is heat sensitive (e.g. degrades or denatures on heating). Suitable ‘cold form’ sealing methods are conducted at a temperature in the range of 150-250° C., more preferably, 210-240° C.

The plural elongate form medicament carriers may be provided to the dispenser in any suitable configuration. One preferred configuration is the ‘side-by-side’ configuration, in which for example, two carriers (e.g. two coiled blister strips) are arranged to lie in sideways alignment with each other in the dispenser. Another preferred configuration is the ‘double-decker’ configuration, in which for example, two carriers (e.g. two coiled blister strips sharing the same coiling axis) are arranged to lie one on top of each other in the dispenser.

The plural carriers are typically provided to the dispenser as separate entities. Alternative embodiments are however, envisaged in which the separate plural elongate carriers are joined together in some appropriate fashion. Thus, for example in a variation of an embodiment comprising two separate elongate strip form carriers each carrying multiple distinct medicament dose portions arranged in series along the respective strip and mountable in the dispenser in ‘double-decker’ configuration there might be provided a single strip comprising two separate series of multiple distinct medicament dose portions arranged in ‘double decker’ configuration (i.e. parallel to each other) as if the two strips of the first embodiment had simply been joined together along adjoining elongate sides thereof.

In a particular ‘joined together’ configuration, two elongate strip form carriers are arranged in ‘back-to-back’ configuration (i.e. one strip backs onto the other with the pockets of each facing outwards). In this embodiment, the ‘back-to-back’ conjoined strip typically has pockets arranged to alternate—one on its first side, then one on the other side. It will be appreciated that when so joined together, each component foil strip of the conjoined whole effectively acts as a ‘lid foil’ for the other.

In one aspect, the elongate form carrier is arranged to have a continuous loop form such as may be achieved by joining the lead end of the strip to the tail end. The loop may be linearly formed or it may be formed as a Mobius strip.

In a particular aspect where the elongate form carrier is in the form of a peelable strip, the base sheet is formed as a continuous loop. In variations, the lid sheet, which forms a peelable sealing lid to the base sheet, may either have continuous loop or non-continuous loop form.

The dispenser has a dispensing mechanism for dispensing the distinct medicament dose portions carried by each of said plural medicament carriers for administration as a single, combination product dose by the patient.

In aspects, some or all components of the dispensing mechanism are common for each of the medicament carriers. The advantage of having common components is that the number of separate parts in the dispenser may be minimised.

In other aspects, the action of those components that are not common may in aspects, be suitably coupled. Coupling is achieved by any suitable fashion including mechanical linkages (e.g. co-gearing or via the use of coupling arms/rods) or electromechanical coupling controls. The advantage of coupling is that the indexing/advancement of each medicament carrier may be achieved in coupled fashion.

In other aspects, most or even all of the components of the dispensing mechanism are distinct. In one particular aspect, the dispenser is arranged such that each of the plural medicament carriers can be indexed/advanced separately thereby providing the opportunity for complex dosing patterns in which any combination, or indeed any one, of the plural strips may be accessed. Where separate indexing/advancement is envisaged separate actuation means (e.g. levers or buttons) may be provided to the dispenser to enable separate actuation thereof.

The mechanism comprises a receiving station for receiving each of the plural medicament carriers. Embodiments are envisaged both in which there is a single receiving station which is capable of receiving plural medicament carriers and also those in which each medicament carrier is received by a distinct (i.e. individual) receiving station. In the latter case, the individual receiving stations may either be coupled or not.

The mechanism further comprises a release for releasing a distinct medicament dose portion from each of the plural medicament carriers on its receipt by the receiving station. The release can have any suitable form. Where the elongate carrier is in the form of a blister strip, the release may for example, comprise means to rupture, puncture, tear or otherwise access the blister. In a particular preferred aspect, where the medicament carrier is in the form of a peelable blister strip the release comprises means for peeling apart the blister strip. In one aspect herein, each blister strip is peeled apart about a defined beak or wedge form feature of the dispenser.

An outlet is positioned to be in communication with the distinct medicament dose portions releasable by said a release to enable their dispensing to the patient. The outlet may have any suitable form. In one aspect, it has the form of a mouthpiece. In another aspect, it has the form of a nozzle for insertion into the nasal cavity of a patient.

The outlet is preferably a single outlet, which communicates with all of the distinct medicament dose portions on their release by said release. Communication is for example, via a common air channeling means (e.g. formed as an air-pipe or common manifold). The patient may therefore breathe in through a single outlet, and that breath be transferred through the common air channeling means to (all of) the released medicament dose portions, thereby enabling their inhalation as a combined product. The outlet and/or channeling device may be shaped to encourage mixing of drug as a result of the airflow created by inhalation by the patient. For example, baffles or other mechanical aids to mixing may be incorporated. Venturi channeling of the airflow is also envisaged in embodiments. Helical form channels are envisaged.

The mechanism also comprises an indexer for indexing (e.g. individually) the distinct medicament dose portions of each of the plural medicament carriers. Said indexing typically happens in sequential fashion, for example accessing dose portions sequentially arranged in series along the length of the elongate carrier. The indexing of each carrier may be arranged to occur in coupled fashion, that is to say each is indexed concurrently.

In a preferred aspect, the medicament carrier comprises a peelable blister strip. In this aspect, the release suitably comprises a peeler for peeling apart a base sheet and lid sheet of each peelable strip to open a pocket. Suitably, the peeler includes lid driver for pulling apart a lid sheet from a base sheet of a pocket that has been received at the opening station.

In a preferred MDPI dispenser aspect, there is provided a medicament dispenser for use with plural blister strip form medicament carriers, each having multiple distinct pockets for containing medicament dose portions, wherein said pockets are spaced along the length of and defined between two peelable sheets secured to each other, said dispenser having a dispensing mechanism for dispensing the medicament dose portions contained within said plural medicament carriers, said mechanism comprising,

a) an opening station for receiving a pocket of each of said medicament carriers;

b) a peeler positioned to engage a base sheet and a lid sheet of a pocket which has been received in said opening station for peeling apart such a base sheet and lid sheet, to open such a pocket;

c) an outlet, positioned to be in communication with an opened pocket through which a user can access a medicament dose portion from such an opened pocket; and

d) an indexer for individually indexing the distinct pockets of each of the plural medicament carriers.

In one aspect, a common opening station is provided for receiving a pocket of each of said medicament carriers. In another aspect, distinct opening stations are provided for receiving a pocket of each medicament carrier. Suitably, the distinct opening stations are linking by a communicating passageway or other means for enabling the coming together of the separately released medicaments.

In the dispenser, each peelable strip form medicament carrier is acted on by a peeler (i.e. peeling means). The peeler engages a base sheet and a lid sheet of a pocket that has been received at the opening station(s) for peeling apart the base sheet and lid sheet to open a pocket. In one aspect, each peelable strip form medicament carrier is acted on by common peeler. In other aspects, each peelable strip is acted on by its own (i.e. separate) peeler.

Suitably, the peeler includes a lid driver for pulling apart a lid sheet and a base sheet of a pocket that has been received at the opening station.

In one aspect, the lid driver comprises a wheel on which the lid sheet is wound up, said wheel having a effective winding surface which remains approximately constant when tension in the lid sheet increases. In one aspect, this is achievable by fashioning the lid driver in ‘collapsible wheel’ form wherein the wheel collapses (i.e. the diameter of the wheel itself decreases) as lid sheet becomes wound around it to give it an overall approximately constant effective winding diameter (as defined by the diameter of the wheel and the strip wound around it). Suitably, said ‘collapsible wheel’ comprises a plurality of resiliently flexible arms each extending there from at an angle with respect to a radius. The leading end of the lid sheet is looped over one of said resiliently flexible arms to secure the lid sheet to the wheel initially.

Alternatively, the lid driver comprises a wheel on which the lid sheet is wound up, said lid sheet wheel having an effective winding surface, the effective diameter of which increases after every use of the dispenser as the lid sheet winds around the wheel. Compensation means are then provided to compensate for this increase, which would otherwise lead to a variation in the tension experienced by the lid sheet over its length and hence a variation in its indexing over time.

In one aspect, there is provided a controller comprising means to limit the extent of movement of said lid driver, in order to control the length of medicament carrier peeled by said peeler. Hence, the medicament carrier is indexed by the same amount each time.

In another aspect, the dispenser comprises compensating means positioned between said opening station and said lid sheet wheel for reducing the length of said lid sheet therebetween to compensate for any increase in the diameter of the effective winding surface of the lid sheet wheel during use of the dispenser.

Suitably, the compensating means takes the form of a flexible member. The flexible member may take the form of a flexible elongate arm about which the lid sheet is fed. The arm may flex inwards as tension in the lid sheet increases, and thus shorten the length of lid sheet between the opening station and the lid driver.

Suitably, the flexible member is resilient so that on removal of tension from the lid sheet, the flexible member returns to its rest position. Thus, the internal mechanism can be reloaded with a new medicament carrier after the used carrier is removed.

In one aspect, the compensating means takes the form of a spring that reduces in length as tension increase in the lid sheet between the opening station and the lid driver. Typically a piston head is mounted on one end of the spring about which the lid sheet is fed. The other end of the spring may be fixed. As tension in the lid sheet increases the piston is driven down onto the spring. Preferably, the compensating means takes the form of a sprung-loaded tensioner.

In another aspect, the compensating means takes the form of a torsion spring mounted at the lid driver that provides compensating torsional force to the lid driver such that the tension provided at the lid sheet remains approximately constant over the length of the blister strip.

Alternatively, or in addition, the dispenser comprises a clutch to adjust for any increase in the diameter of the effective winding surface of the lid driver during use of the dispenser. In one aspect, the clutch communicates with the an indexer and the lid driver, and comprises a gearing surface defining plural gear engagement positions; and plural gear teeth for engaging said plural gear engagement positions, wherein the plural gear teeth are arranged such that at any one time only a single gear tooth engages a single gear engagement position.

In use, the clutch acts to compensate for the increase in diameter of said effective winding surface of the lid driver. The clutch allows for slippage when the tension in the lid sheet is greater than the force required to peel apart the lid sheet and the base sheet.

Suitably, the dispenser comprises a guide for guiding the lid sheet and base sheet along separate paths at the opening station. The lid sheet is passed around the guide portion onto the lid driver. In one aspect, the guide comprises a roller mechanism. The lid sheet is fed over the rollers onto the lid driver.

The mechanism includes an indexer for individually indexing the distinct pockets of each of the plural medicament carriers. Suitably, the an indexer comprises a rotatable index wheel having recesses therein, said index wheel being engageable with a medicament carrier in use with said medicament dispenser such that said recesses each receive a respective pocket of the base sheet of a blister strip in use with said medicament dispenser.

Suitably, the rotatable index wheel additionally comprises a series of indentations located at its base and spaced in between the recesses.

Suitably, the indexer additionally comprises an interlock coupling to couple actuation of the dispenser to the index wheel. The interlock coupling reversibly locks the index wheel in place. Preferably, said interlock coupling comprises a foot portion having a toe and a heel, and a tail section. Preferably, said interlock coupling is pivotally mountable to the dispenser at its foot portion. Preferably, said toe fits into one of the indentations on the rotatable index wheel. Preferably, the interlock coupling is sprung to bias it towards location of the toe in one of the indentations.

Alternatively, the indexer comprises a gear and sprocket wherein teeth on the wheel fit into apertures or holes formed on one or both edges of a medicament carrier. The mechanism therefore resembles that of photographic film being advanced through a camera.

Alternatively, the indexer comprises an index ratchet that is moveable between a locked position whereby said ratchet engages a pocket on said medicament carrier and prevents further peeling thereof, and a release position allowing free movement of said medicament carrier. In this embodiment, actuation of said medicament dispenser actuates said lid driver and releases said index ratchet from a medicament carrier to allow peeling thereof.

Suitably, the dispenser additionally comprises a first chamber in which at least one medicament carrier is initially housed and from which it is dispensed and a second chamber to receive the used portion of the base sheet after it has been indexed around the index wheel and separated from the lid sheet. Suitably, said first chamber and said second chamber are separable by a wall. In one aspect, said wall is movable to adjust the size of said first and second chambers. In another aspect, the wall is pivotally mountable. Alternatively the wall is slidably mountable.

Suitably, the internal mechanism further comprises a third chamber to receive the used portion of the lid sheet and a fourth chamber which houses the index ratchet. The fourth chamber may communicate via a slit, which in turn extends upwardly within a mouthpiece and communicates with air inlets.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose portion of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085.

The medicament dispenser device herein has unitary form, and typically comprises a housing shaped to receive, and enable the release of active medicament from the first and second medicament containers.

In one aspect, the housing integrally comprises a release means for releasing medicament from at least one, preferably both medicament containers.

In another aspect, the housing is shaped to receive the medicament containers, each of which is provided with respective release means. In this case, the release means have typically been adapted for receipt by the housing.

Suitably, the release means for each medicament container is coupled, thereby enabling simultaneous delivery of medicament from each container in response to a single patient actuation step.

In one aspect, each of the medicament containers is sized and shaped to deliver equivalent dose portions, that is to say each container is arranged to release dose portions of equivalent dose volume or dose weight. In one particular example, each medicament container is arranged to release plural 12 mg (or 25 mg) dose portions.

In another aspect, each of the medicament containers is sized and shaped to deliver non-equivalent dose portions, that is to say each container is arranged to release dose portions of non-equivalent dose volume or dose weight to the other. In one specific example, the first medicament container is arranged to carry plural 12 mg dose portions and the second container is arranged to carry plural 25 mg dose portions.

In aspects, any or all components of the medicament dispenser herein may be driven by either an electronic or mechanical drive system or combination thereof.

Suitably electronic drive means typically comprise a motor, preferably an electrically powered motor. The motor may provide linear or rotary drive, but in general, rotary motors are most suitable. The motor may for example, comprise a DC electric motor, a piezoelectric (PZ) motor, an ultrasonic motor, a solenoid motor or a linear motor. Preferably, the electronic drive system comprises a DC motor, a PZ motor or an ultrasonic motor.

The use of ultrasonic motors is particularly preferred since they offer advantages over conventional motors in terms of weight, size, noise, cost and torque generated. Ultrasonic motors are well known in the art and are commercially available (e.g. BMSTU Technological Cooperation Centre Ltd, Moscow, Russia; Shinsei Corporation, Tokyo, Japan).

Ultrasonic motors do not use coils or magnets but comprise a piezo-electric ceramic stator that drives a coupled rotor. The stator generates ultrasonic vibrations, which in turn causes rotation of the rotor. While regular DC motors are characterised by high speed and low torque, requiring reduction gearing to increase torque, ultrasonic motors attain low speed and high torque, thus eliminating the need for reduction gearing. Furthermore, these motors are lightweight and compact, lacking coils and magnets, and are noiseless as the ultrasonic frequencies used are not audible to the human ear.

Suitably, the medicament dispenser further comprises actuating means for actuating a manual or electronic drive system. Said actuating means may take the form of a switch, push-button, or lever.

In one aspect, the medicament dispenser herein is configured to be reloadable. In particular, each medicament container is suitably provided in refill form or alternatively, within a refill cassette. Suitably, each refill is independently accessible/operable.

In one aspect, each medicament container is provided as a separate refill. In another aspect, both medicament containers are provided in tandem (e.g. within a single refill cassette).

In particular aspect, the medicament dispenser herein is configured to comprise a body; a holder, shaped to fit within said body and movable relative to said body; and receivable by said holder, a cassette containing the first and second medicament containers.

Suitably, any drive system (e.g. electronic) is located in either the body or the holder part, and the cassette comprises the minimum number of component (i.e. internal mechanism) parts. In embodiments, the body/holder including the (e.g. electronic) drive is retainable by the user and the cassette is sold as a refill/reload component that is discarded after use. By locating the drive system in the body/holder, the amount of components that are discarded with a spent refill cassette is minimised which is beneficial from an environmental standpoint.

Suitably, the holder includes guide means to guide the cassette into the holder. Preferably said guide means comprise guide rails. Alternatively the guide means comprise grooves, indentations or other shaping or surface details to define a ‘lock and key’ relationship between the holder and the cassette. Colour guides, arrows and any other surface markings may also be employed.

Suitably, the cassette additionally comprises means to actuate the dispenser. The actuating means may take the form of a switch, push-button or lever.

In one MDPI dispenser aspect, a refill cassette for the medicament dispenser herein comprises

a) an opening station for receiving a pocket of each of the plural form medicament carriers;

b) a peeler positioned to engage a base sheet and a lid sheet of a pocket which has been received in said opening station for peeling apart such a base sheet and lid sheet, to open such a pocket;

c) an outlet, positioned to be in communication with an opened pocket through which a user can access a medicament dose portion from such an opened pocket; and

d) an indexer for individually indexing the distinct pockets of each of the plural medicament carriers.

The medicament dispenser herein may be designed for nasal inhalation of a powdered medicament and may therefore incorporate a nozzle as an alternative to a mouthpiece. If the medicament is in solid form, the dispenser may incorporate an exit channel for tablet release.

The medicament dispenser in reloadable form may be supplied as a kit of parts. A first part of the kit comprises a body; a holder, shaped to fit within said body and movable relative to said body; and within said holder a receiving station for receipt of a cassette. A second part of the kit comprises a cassette containing plural medicament containers and a dispensing mechanism for dispensing from said plural medicament containers, wherein the cassette is receivable by the receiving station and movement of the holder relative to the body results in movement of the cassette between a first position and a second position such that the cassette is reversibly removable from the receiving station when the cassette is in the second position.

In one aspect, the reloadable dispenser is assembled as follows. The holder is snap fitted into the body. The cassette is assembled separately. The body of the cassette is formed, preferably in two sections with any further components formed into the base. Other individual components are then assembled into the base. Finally the plural medicament containers (e.g. MDI canisters or DPI blister packs) are inserted into the cassette. Alternatively, the cassette may be formed completely apart from a hole left in its side for insertion of the medicament containers. The hole may then be sealed to complete the cassette. This second method of inserting the medicament containers into the device has the advantage that it is much simpler.

Suitably, the medicament dispenser herein comprises an actuation or dose counter for counting the number of actuations of the indexing lever or releases of dose from the cassette. The dose counter may count the number of doses left to be taken or the number of doses taken. In one aspect, the dose counter is electronic. Alternatively said dose counter is mechanical.

Suitably, the medicament dispenser additionally includes an electronic data management system. The electronic data management system has input/output capability and comprises a memory for storage of data; a microprocessor for performing operations on said data; and a transmitter for transmitting a signal relating to the data or the outcome of an operation on the data.

Suitably, the electronic data management system is arranged to be responsive to or activated by the voice of a user. Thus, for example the system may be switched on or off in response to a voice command.

The electronic data management system may be integral with the body of the dispenser. Alternatively, the electronic data management system forms part of a base unit which is reversibly associable with the body.

Suitably, the medicament dispenser additionally comprises a data input system for user input of data to the electronic data management system. Preferably, the data input system comprises a man machine interface (MMI) preferably selected from a keypad, voice recognition interface, graphical user interface (GUI) or biometrics interface.

Energy may be conserved by a variety of means to enable the dispenser to operate for longer on a given source of energy, such as a battery. Energy conservation or saving methods have additional advantages in terms of reducing the size requirements of the power source (e.g. battery) and thus the weight and portability of the medicament dispenser.

A variety of energy saving methods is available which generally involve reducing power consumption. One such method is to use a clock or timer circuit to switch the power on and off at regular or predetermined intervals. In another method the system can selectively switch on/off specific electronic devices, such as visual display units or sensors, in order to power these devices only when they are required to perform a particular sequence of events. Thus different electronic devices may be switched on and off at varying intervals and for varying periods under control of the system. The power sequencing system may also respond to a sensor, such as a motion or breath sensor, which is activated on use of the device.

Low power or “micropower” components should be used within the electronics where possible and if a high power device is required for a particular function this should be put into a low power standby mode or switched off when not required. Similar considerations apply in the selection of transducers. Operation at low voltage is desirable since power dissipation generally increases with voltage.

For low power digital applications complementary metal oxide semi-conductor (CMOS) devices are generally preferred and these may be specially selected by screening for low quiescent currents. Clock speeds of processors and other logic circuits should be reduced to the minimum required for computational throughput as power consumption increases with frequency. Supply voltages should also be kept at minimal values consistent with reliable operation because power dissipation in charging internal capacitance's during switching is proportional to the square of the voltage. Where possible, supply voltages should be approximately the same throughout the circuit to prevent current flowing through input protection circuits. Logic inputs should not be left floating and circuits should be arranged so that power consumption is minimised in the most usual logic output state. Slow logic transitions are undesirable because they can result in relatively large class-A currents flowing. Resistors may be incorporated in the power supply to individual devices in order to minimise current in the event of failure.

In some control applications, devices that switch between on and off states are preferred to those that allow analog (e.g. linear) control because less power is dissipated in low resistance on states and low current off states. Where linear components are used (e.g. certain types of voltage regulators) then types with low quiescent currents should be selected. In some circuit configurations it is preferable to use appropriate reactive components (i.e. inductors and capacitors) to reduce power dissipation in resistive components.

Suitably, the system additionally comprises a visual display unit for display of data from the electronic data management system to the user. The display may for example, comprise a screen such as an LED or LCD screen. More preferably the visual display unit is associable with the body of the medicament dispenser.

Suitably, the medicament dispenser additionally comprises a datalink for linking to a local data store to enable communication of data between the local data store and the electronic data management system. The datastore may also comprise data management, data analysis and data communication capability.

The datastore may itself form part of a portable device (e.g. a handheld device) or it may be sized and shaped to be accommodated within the patient's home. The datastore may also comprise a physical storage area for storage of replacement cassettes. The datastore may further comprise a system for refilling medicament from a reservoir of medicament product stored therewithin. The datastore may further comprise an electrical recharging system for recharging any electrical energy store on the medicament dispenser, particularly a battery recharging system.

The datalink may for example enable linking with a docking station, a personal computer, a network computer system or a set-top box by any suitable method including a hard-wired link, an infrared link or any other suitable wireless communications link.

Suitably, the medicament dispenser additionally comprises an actuation detector for detecting actuation of the dispensing mechanism wherein said actuation detector transmits actuation data to the electronic data management system.

The medicament dispenser may additionally comprise a safety mechanism to prevent unintended multiple actuations of the dispensing mechanism. The patient is thereby protected from inadvertently receiving multiple doses of medicament in a situation where they take a number of short rapid breaths. More preferably, the safety mechanism imposes a time delay between successive actuations of the release. The time delay is typically of the order of from three to thirty seconds.

Suitably, the medicament dispenser additionally comprises a release detector for detecting release of medicament, wherein said release detector transmits release data to the electronic data management system.

Suitably, the medicament dispenser additionally comprises a shake detector for detecting shaking of the medicament containers (e.g. prior to actuation of the dispensing mechanism), wherein said shake detector transmits shake data to the electronic data management system.

Suitably, any actuation detector, release detector, or shake detector comprises a sensor for detecting any suitable parameter such as movement. Any suitable sensors are envisaged including the use of optical sensors. The release detector may sense any parameter affected by release of the medicament such as pressure, temperature, sound, moisture, carbon dioxide concentration and oxygen concentration.

Suitably, the medicament dispenser additionally comprises a breath trigger for triggering the dispensing mechanism, said breath trigger being actuable in response to a trigger signal from the electronic data management system. Preferably, the electronic data management system includes a predictive algorithm or look-up table for deriving from the breath data when to transmit the trigger signal. For example, a real-time analysis of the patient breath waveform may be made and the trigger point derived by reference to that analysed waveform.

Suitably, the electronic data management system includes a predictive algorithm or look-up table for calculating the optimum amount of medicament to dispense.

Suitably, the memory on the electronic data management system includes a dose memory for storing dosage data and reference is made to the dose memory in calculating the optimum amount of medicament to dispense.

Suitably, the medicament dispenser additionally comprises a selector for selecting the amount of medicament to dispense from said dispensing mechanism. In one aspect, the selector is manually operable. In another aspect, the selector is operable in response to a signal from the transmitter on the electronic data management system.

Suitably, the medicament dispenser comprises in association with a body or housing thereof, a first transceiver for transmitting and receiving data and in association with each medicament container, a second transceiver for transmitting and receiving data, wherein data is transferable in two-way fashion from the first transceiver to the second transceiver. The data is preferably in digital form and suitable for transfer by electronic or optical means.

One advantage of embodiments of this type is the ability to store many types of information in different parts of the memory structure of the transceivers. The information is furthermore stored in a form which is readily and accurately transferable. The information could for example, include manufacturing and distribution compliance information written to the memory at various points in the manufacturing or distribution process, thereby providing a detailed and readily accessible product history of the dispenser. Such product history information may, for example, be referred to in the event of a product recall. The compliance information could, for example, include date and time stamps. The information could also include a unique serial number stored in encrypted form or in a password protectable part of the memory which uniquely identifies the product and therefore may assist in the detection and prevention of counterfeiting. The information could also include basic product information such as the nature of the medicament and dosing information, customer information such as the name of the intended customer, and distribution information such as the intended product destination.

On loading or reloading the medicament dispenser with a cassette the second transceiver may, for example, read the unique serial number, batch code and expiry date of the medicament and any other information on the second transceiver. In this way the nature and concentration of the medicament, together with the number of doses used or remaining within the cassette, may be determined. This information can be displayed to the patient on a visual display unit. Other information, such as the number of times the medicament dispenser has been reloaded with a cassette, may also be displayed.

Similarly, should the cassette be removed from the holder before the supply of medicament is exhausted, the same data can be read from the second transceiver and the number of doses remaining or used determined. Other information, such as the date and time of administration of the drug, or environmental exposure data such as the minimum/maximum temperatures or levels of humidity the cassette has been exposed to, may also be read and displayed to the user.

In the event that the supply of medicament within each container becomes exhausted, or that the shelf life of the medicament has expired, or that the first transceiver does not recognise the batch code on the second transceiver, activation of the dispenser may be prevented to safeguard the user. Activation may also be prevented if the medicament has been exposed to extreme environmental conditions for periods outwith the manufacturer's guidelines.

Data may be transferred to and from any transceiver during the period of use of the medicament dispenser by the patient. For example, the medicament dispenser may include an electronic data management system having various sensors associated therewith. Any data collected by the sensors or from any data collection system associated with the electronic data management system including a clock or other date/time recorder is transferable.

Data may be transferred each time the patient uses the dispenser. Or alternatively, data may be stored in a database memory of the electronic data management system and periodically downloaded to any transceiver. In either case, a history of the usage of the dispenser may be built up in the memory of a transceiver.

In one embodiment herein, a history of the usage of the medicament dispenser is transferred to the second transceiver. When the medicament carriers in the cassette are exhausted, the cassette is exchangeable by the patient for a new refill cassette. At the point of exchange, which will typically occur at the pharmacy, data may be transferred from the exhausted cassette to the refill and vice-versa. Additionally, usage history data may be read from the refill and transferred to a healthcare data management system for example comprising a network computer system under the control of a healthcare data manager.

Methods are envisaged herein whereby the patient is given some sort of reward for returning the refill and making available the data comprised within the second transceiver. Methods are also envisaged herein whereby the healthcare data manager is charged for either receipt of the data from the second transceiver or for its use for commercial purposes. Any rewards or charging may be arranged electronically. The methods may be enabled by distributed or web-based computer network systems in which any collected data is accessible through a hub on the network. The hub may incorporate various security features to ensure patient confidentiality and to allow selective access to information collected dependent upon level of authorisation. The level of user authorisation may be allocated primarily to safeguard patient confidentiality. Beyond this the level of user authorisation may also be allocated on commercial terms with for example broader access to the database being authorised in return for larger commercial payments.

Suitably, the first and second transceiver each comprise an antenna or equivalent for transmitting or receiving data and connecting thereto a memory. The memory will typically comprise an integrated circuit chip. Either transceiver may be configured to have a memory structure which allows for large amounts of information to be stored thereon. The memory structure can be arranged such that parts of the memory are read-only, being programmed during/after manufacture, other parts are read/write and further parts are password protectable. Initial transfer of information (e.g. on manufacture or one dispensing) to or from any transceiver can be arranged to be readily achievable by the use of a reader which is remote from the medicament dispenser, thereby minimising the need for direct product handling. In further aspects, the reader can be arranged to simultaneously read or write to the memory of multiple transceivers on multiple medicament dispensers.

A suitable power source such as a battery, clockwork energy store, solar cell, fuel cell or kinetics-driven cell will be provided as required to any electronic component herein. The power source may be arranged to be rechargeable or reloadable.

Suitably, data is transferable in two-way fashion between the first and second transceiver without the need for direct physical contact therebetween. Preferably, data is transferable wirelessly between the first and second transceiver.

Suitably, the first transceiver is an active transceiver and the second transceiver is a passive transceiver. The term active is used to mean directly-powered and the term passive is used to mean indirectly-powered.

Suitably, the second transceiver comprises a label or tag comprising an antenna for transmitting or receiving energy; and an integrated circuit chip connecting with said antenna, and the first transceiver comprises a reader for said label or tag. In this case the label or tag is a passive transceiver and the reader is an active transceiver. Preferably, the reader will not need to be in direct contact with the tag or label to enable the tag or label to be read.

The tag may be used in combination and/or integrated with other traditional product labelling methods including visual text, machine-readable text, bar codes and dot codes.

Suitably, the integrated circuit chip has a read only memory area, a write only memory area, a read/write memory area or combinations thereof.

Suitably, the integrated circuit chip has a one-time programmable memory area. More preferably, the one-time programmable memory area contains a unique serial number.

Suitably, the integrated circuit chip has a preset memory area containing a factory preset, non-changeable, unique data item. The preset memory item is most preferably in encrypted form.

Suitably, the integrated circuit chip has plural memory areas thereon. Suitably, any memory area is password protected.

Suitably, any memory area contains data in encrypted form. Electronic methods of checking identity, error detection and data transfer may also be employed.

In one aspect, the integrated circuit has plural memory areas thereon including a read only memory area containing a unique serial number, which may for example be embedded at the time of manufacture; a read/write memory area which can be made read only once information has been written thereto; and a password protected memory area containing data in encrypted form which data may be of anti-counterfeiting utility.

Suitably, the tag is on a carrier and the carrier is mountable on the body or holder of the medicament dispenser or on the cassette.

In one aspect, the carrier is a flexible label. In another aspect, the carrier is a rigid disc. In a further aspect, the carrier is a rectangular block. In a further aspect, the carrier is a collar ring suitable for mounting to the neck of an aerosol container. Other shapes of carrier are also envisaged.

Suitably, the carrier is moldable or weldable to the cassette or housing. Suitably, the carrier encases the tag. More preferably, the carrier forms a hermetic seal for the tag. In one aspect, the carrier comprises an insulating material such as a glass material or, a paper material or an organic polymeric material such as polypropylene. Alternatively, the carrier comprises a ferrite material.

The energy may be in any suitable form including ultrasonic, infrared, radiofrequency, magnetic, optical and laser form. Any suitable channels may be used to channel the energy including fibre optic channels.

In one aspect, the second transceiver comprises a radiofrequency identifier comprising an antenna for transmitting or receiving radiofrequency energy; and an integrated circuit chip connecting with said antenna, and the first transceiver comprises a reader for said radiofrequency identifier. In this case the radiofrequency identifier is a passive transceiver and the reader is an active transceiver. An advantage of radiofrequency identifier technology is that the reader need not be in direct contact with the radiofrequency identifier tag or label to be read.

The radiofrequency identifier can be any known radiofrequency identifier. Such identifiers are sometimes known as radiofrequency transponders or radiofrequency identification (RFID) tags or labels. Suitable radiofrequency identifiers include those sold by Phillips Semiconductors of the Netherlands under the trade marks Hitag and Icode, those sold by Amtech Systems Corporation of the United States of America under the trade mark Intellitag, and those sold by Texas Instruments of the United States of America under the trade mark Tagit.

Suitably, the antenna of the RFID tag is capable of transmitting or receiving radiofrequency energy having a frequency of from 100 kHz to 2.5 GHz. Preferred operating frequencies are selected from 125 kHz, 13.56 MHz and 2.4 GHz.

In one aspect, the second transceiver comprises a magnetic label or tag comprising an antenna for transmitting or receiving magnetic field energy; and an integrated circuit chip connecting with said antenna, and the first transceiver comprises a reader for said magnetic label or tag. In this case the magnetic label or tag is a passive transceiver and the reader is an active transceiver.

A suitable magnetic label or tag comprises plural magnetic elements in mutual association whereby the magnetic elements move relative to each other in response to an interrogating magnetic field. A magnetic label or tag of this type is described in U.S. Pat. No. 4,940,966. Another suitable magnetic label or tag comprises a magnetorestrictive element which is readable by application of an interrogating alternating magnetic field in the presence of a magnetic bias field which results in resonance of the magnetorestrictive elements at different predetermined frequencies. A magnetic label of this type is described in PCT Patent Application No. WO92/12402. Another suitable magnetic label or tag comprising plural discrete magnetically active regions in a linear array is described in PCT Patent Application No. WO96/31790. Suitable magnetic labels and tags include those making use of Programmable Magnetic Resonance (PMR) (trade name) technology.

In another aspect, the second transceiver comprises a microelectronic memory chip and the first transceiver comprises a reader for said microelectronic memory chip. The microelectronic memory chip may comprise an Electrically Erasable Programmable Read Only Memory (EEPROM) chip or a SIM card-type memory chip. In this case the microelectronic memory chip is a passive transceiver and the reader is an active transceiver.

Any transceiver herein, particularly a passive transceiver may be mounted on or encased within any suitable inert carrier. The carrier may comprise a flexible sheet which may in embodiments be capable of receiving printed text thereon.

In one aspect, the first transceiver is integral with the body such that a single unit is comprised. The first transceiver may for example be encased within or moulded to the body.

In another aspect, the first transceiver forms part of a base unit which is reversibly associable with the body. The base unit may for example, form a module receivable by the body such as a snap-in module.

Suitably, the medicament dispenser additionally comprises a communicator for wireless communication with a network computer system to enable transfer of data between the network computer system and the electronic data management system. Preferably, the communicator enables two-way transfer of data between the network computer system and the electronic data management system.

Suitably, the data is communicable between the network computer system and the electronic data management system in encrypted form. All suitable methods of encryption or partial encryption are envisaged. Password protection may also be employed. Suitably, the communicator employs radiofrequency or optical signals.

In one aspect, the communicator communicates via a gateway to the network computer system. In another aspect, the communicator includes a network server (e.g. a web server) such that it may directly communicate with the network.

In a further aspect, the communicator communicates with the gateway via a second communications device. Preferably, the second communications device is a telecommunications device, more preferably a cellular phone or pager. Preferably, the communicator communicates with the second communications device using spread spectrum radiofrequency signals. A suitable spread spectrum protocol is the Bluetooth (trade mark) standard which employs rapid (e.g. 1600 times a second) hopping between plural frequencies (e.g. 79 different frequencies). The protocol may further employ multiple sending of data bits (e.g. sending in triplicate) to reduce interference.

In one aspect, the network computer system comprises a public access network computer system. The Internet is one suitable example of a public access network computer system, wherein the point of access thereto can be any suitable entrypoint including an entrypoint managed by an Internet service provider. The public access network computer system may also form part of a telecommunications system, which may itself be either a traditional copper wire system, a cellular system or an optical network.

In another aspect, the network computer system comprises a private access network computer system. The private access network system may for example, comprise an Intranet or Extranet that may for example, be maintained by a health service provider or medicament manufacturer. The network may for example include password protection; a firewall; and suitable encryption means.

Preferably, the communicator enables communication with a user-specific network address in the network computer system.

The user-specific network address may be selected from the group consisting of a web-site address, an e-mail address and a file transfer protocol address. Preferably, the user-specific network address is accessible to a remote information source such that information from said remote information source can be made available thereto. More preferably, information from the user-specific network address can be made available to the remote information source.

In one aspect, the remote information source is a medicament prescriber, for example a doctors practice. Information transferred from the medicament prescriber may thus, comprise changes to prescription details, automatic prescription updates or training information. Information transferred to the medicament prescriber may comprise compliance information, that is to say information relating to the patient's compliance with a set prescribing programme. Patient performance information relating for example, to patient-collected diagnostic data may also be transferred to the medicament prescriber. Where the dispenser is an inhaler for dispensing medicament for the relief of respiratory disorders examples of such diagnostic data would include breath cycle data or peak flow data.

In another aspect, the remote information source is a pharmacy. Information transferred from the pharmacy may thus, comprise information relating to the medicament product. Information sent to the pharmacy may thus include prescription requests which have been remotely pre-authorised by the medicament prescriber.

In a further aspect, the remote information source is an emergency assistance provider, for example a hospital accident and emergency service or an emergency helpline or switchboard. The information may thus, comprise a distress or emergency assist signal which requests emergency assistance.

In a further aspect, the remote information source is a manufacturer of medicament or medicament delivery systems. Information transferred to the system may thus, comprise product update information. The system may also be configured to feed information back to the manufacturer relating to system performance.

In a further aspect, the remote information source is a research establishment. In a clinical trial situation, information may thus be transferred relating to the trial protocol and information relating to patient compliance fed back to the research establishment.

In a further aspect, the remote information source is an environmental monitoring station. Information relating to weather, pollen counts and pollution levels may thus be made accessible to the system.

Suitably, the medicament dispenser additionally comprises a geographic positioning system such as a global positioning system or a system that relies on the use of multiple communications signals and a triangulation algorithm.

In one aspect, the medicament dispenser herein does not comprise an electronic control system for controlling the release of contents from the first and second medicament containers. In one aspect, the medicament dispenser herein does not contain an actuation indicator associated with the first and second medicament containers.

According to another aspect of the present invention there is provided the use of the dispenser herein for dispensing a combination medicament product.

### BRIEF DESCRIPTION OF THE DRAWINGS

The invention will now be described with reference to the accompanying drawings in which:

FIG. 1 shows a perspective view of an elongate form medicament carrier suitable for use in accord with an MDPI dispenser of the present invention;

FIG. 2a shows a sectional plan view of a first MDPI dispenser in accord with the invention;

FIG. 2b shows a perspective view of a detail of the MDPI dispenser of FIG. 2a;

FIG. 3 shows a sectional plan view of a second MDPI dispenser in accord with the invention;

FIG. 4a shows a sectional plan view of a third MDPI dispenser in accord with the invention;

FIG. 4b shows a blown apart, perspective view of the MDPI dispenser of FIG. 4a in which two medicament carrier strips associated therewith are shown removed from the dispenser;

FIG. 5a shows a sectional plan view of a fourth MDPI dispenser in accord with the invention;

FIG. 5b shows a blown apart, perspective view of the MDPI dispenser of FIG. 5a in which the strip form, dual series medicament carrier associated therewith is shown removed from the dispenser;

FIGS. 6a and 6b respectively show schematic top and bottom views of a reservoir DPI dispenser in accord with the present invention, wherein the mouthpiece is in the storage position;

FIGS. 6c and 6d respectively show schematic top and bottom views of the reservoir DPI dispenser of FIGS. 6a and 6b, wherein the mouthpiece is in the in-use position;

FIGS. 7a and 7b respectively show upper and lower sectional side views of a second reservoir DPI dispenser in accord with the present invention, wherein the mouthpiece is in the storage position;

FIGS. 7c and 7d respectively show upper and lower sectional side views of the reservoir DPI dispenser of FIGS. 7a and 7b, wherein the mouthpiece is in the in-use position;

FIGS. 8a to 8c show a third reservoir DPI dispenser herein respectively in perspective, exploded (part cut-away) and sectional side views;

FIGS. 9a to 9c show a first UDPI capsule dispenser herein respectively in perspective, exploded and sectional side views;

FIGS. 10a to 10c show a second UDPI capsule dispenser herein respectively in perspective, exploded and sectional side views;

FIGS. 11a and 11b show a fourth MDPI dispenser herein respectively in exploded perspective and side views;

FIGS. 12a and 12b show a fifth MDPI dispenser herein respectively in perspective and exploded top views; and

FIG. 13a shows a perspective view of a dual MDI dispenser herein and FIG. 13b shows the dispenser of FIG. 13a in part cut-away view.

### DETAILED DESCRIPTION OF THE DRAWINGS

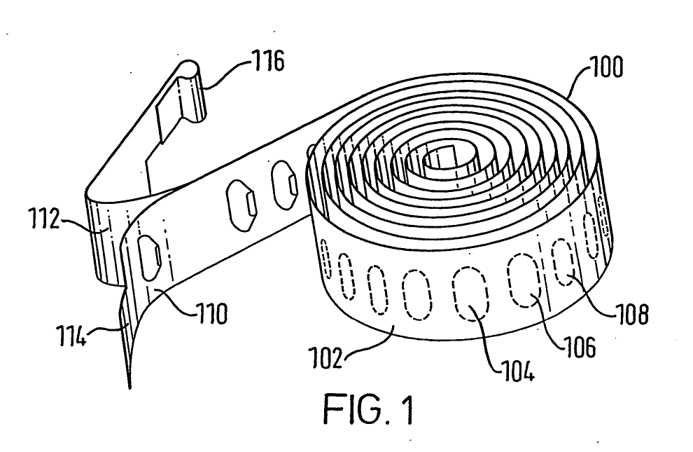

The Figures herein show details of suitable medicament dispensers (with the exception of FIG. 1 which shows a suitable medicament container) in accord with the invention. In use, the first medicament container of each dispenser comprises a first medicament active component selected from the group consisting of salmeterol, formoterol and any salts or solvates thereof; and the second medicament container of each dispenser comprises a second medicament active component selected from the group consisting of beclomethasone ester, fluticasone ester and any salts or solvates thereof.

FIG. 1 shows a medicament carrier 100 suitable for use in an MDPI type dispenser in accord with the present invention. The medicament carrier comprises a flexible strip 102 defining a plurality of pockets 104, 106, 108 each of which contains a portion of a dose of active medicament of a form suitable for inhalation and in the form of powder. In accord with the present invention, two such strips 102, one containing the first active medicament and the other containing the second active medicament are employed in a single medicament dispenser, wherein each strip provides the component active medicament dose portions of the combination medicament product. Each strip may be of the same size and/or contain the same dose amount (e.g. volume or mass) or in alternative embodiments, strips of different sizes and/or containing different dose amounts may be employed in combination.

The strip comprises a base sheet 110 in which blisters are formed to define the pockets 104, 106, 108 and a lid sheet 112 which is hermetically sealed to the base sheet except in the region of the blisters in such a manner that the lid sheet 112 and the base sheet 110 can be peeled apart. The sheets 110, 112 are sealed to one another over their whole width except for the leading end portions 114, 116 where they are preferably not sealed to one another at all.

The lid 112 and base 110 sheets are each formed of a plastics/aluminium laminate and are suitably adhered to one another by heat sealing. The lid sheet 112 comprises at least the following successive layers: (a) paper; adhesively bonded to (b) polyester; adhesively bonded to (c) aluminium foil; that is coated with a heat seal lacquer for bonding to the base sheet. The base sheet 110 comprises at least the following successive layers: (a) oriented polyamide (OPA); adhesively bonded to (b) aluminium foil; adhesively bonded to (c) a third layer comprising a polymeric material (e.g. polyvinyl chloride).

The strip 102 is shown as having elongate pockets 104, 106, 108 which run transversely with respect to the length of the strip 102. This is convenient in that it enables a large number of pockets 104, 106, 108 to be provided in series arrangement along a given strip 102 length. The strip 102 may, for example, be provided with sixty or one hundred pockets but it will be understood that the strip 102 may have any suitable number of pockets.

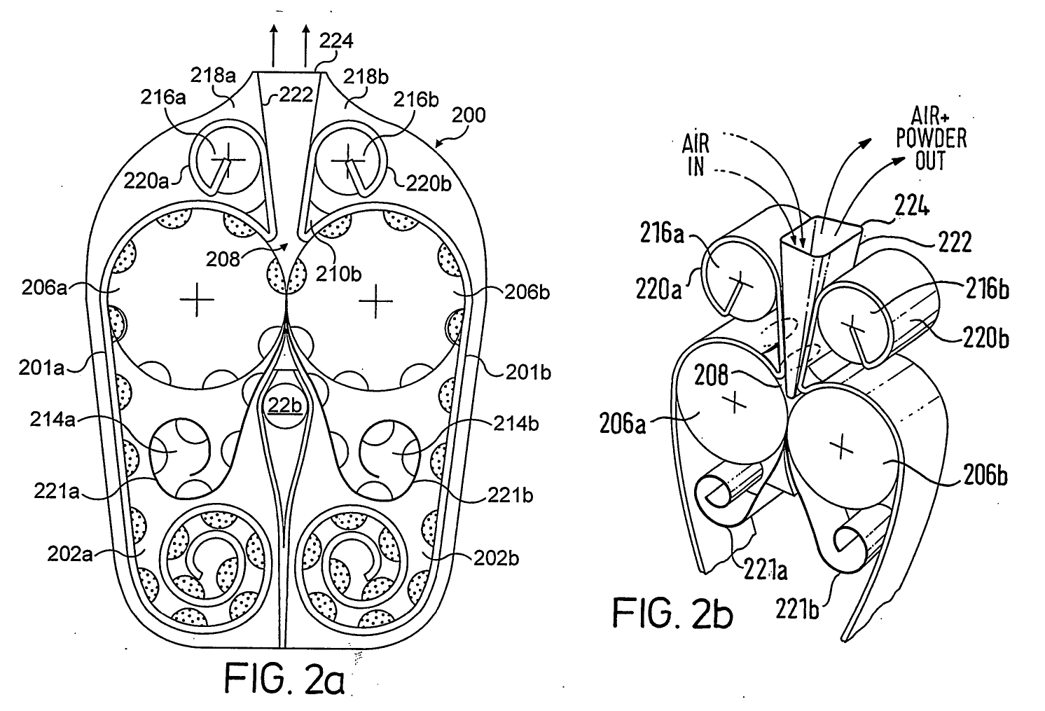

FIG. 2a illustrates the base unit 200 of a medicament dispenser according to the invention. In use, a cover (not shown) would be provided to the base unit 200. First and second medicament-containing blister strips 201a, 201b are positioned within respective left and right chambers 202a, 202b of the base unit 200. The first blister strip 201a contains multiple dose portions of a first active medicament component. The second blister strip 201b contains multiple dose portions of a second active medicament component. Each blister strip 201a, 201b engages a respective multi-pocket index wheel 206a, 206b, and successive pockets are thereby guided towards a commonly located opening station 208. The rotation of the index wheels 206a, 206b is coupled. At the opening station 208, the lid foil 220a, 220b and base foil 221a, 221b parts of each strip 201a, 201b are peelably separable about a beak 210a, 210b. The resulting empty base foil 221a, 221b coils up in respective base take-up chambers 214a, 214b. The used lid foil 220a, 220b is fed over its respective beak 210a, 210b and coiled about a lid take-up spindle 216a, 216b in the lid take-up chamber 218a, 218b.

Released powder form medicament from both the first 201a and second 201b strips is channeled via common manifold 222 to a single outlet 224 for inhalation by the patient. Importantly, the dispenser thereby enables different medicament types to be stored separately in each of the strips 201a, 201b but the release and delivery thereof to the patient as a combined inhaled product.

FIG. 2b shows the release of medicament in more detail. The patient breathes in through the outlet 224 resulting in negative pressure being transmitted through manifold 222 to the opened pockets of the strips 201a, 201b at the opening station 208. This results in the creation of a venturi effect which results in the powder contained within each of the opened pockets 201a, 201b being drawn out through the common manifold 222 to the outlet 224 and hence to the patient. Mixing of each separately delivered component of the combined medicament product will thus happens during the delivery process, particularly as a result of the so created venturi effect.

The dispenser is actuated by pressing a button on the side of the dispenser (not shown) which actuates a DC motor 226 to index the internal mechanism by one pocket of medicament for each blister strip 201a, 201b. The DC motor 226, thus results in indexing of each strip 201a, 201b and coiling up of the waste foils.

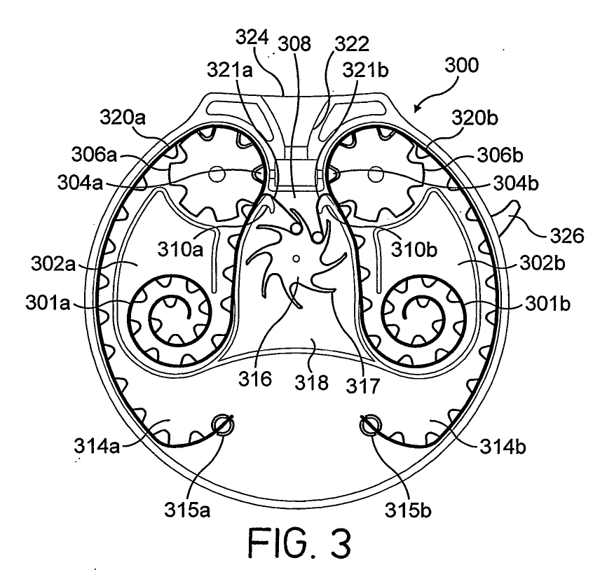

FIG. 3 illustrates a sectional view of base unit 300 of a medicament dispenser according to the invention. In use, a protective cover (not shown) would be provided to the base unit 300. First and second medicament-containing blister strips 301a, 301b are positioned within respective left and right chambers 302a, 302b of the base unit 300. The first blister strip 301a contains multiple dose portions of a first active medicament component. The second blister strip 301b contains multiple dose portions of a second active medicament component. Each blister strip 301a, 301b engages in respective multi-pocket index wheel 306a, 306b, and successive pockets are thereby guided towards a central opening station 308. The rotation of the index wheels 306a, 306b is optionally coupled together. At the opening station 308, the lid foil 320a, 320b and base foil 321a, 321b parts of each strip 301a, 301b are peelably separable about beak 310a, 310b. The resulting empty base foil 321a, 321b coils up in respective base take-up chambers 314a, 314b. A base foil anchor 315a, 315b anchors the end of each respective base foil 321a, 321b in its chamber 314a, 314b. The used lid foil 320a, 320b feeds over its respective beak 310a, 310b and coils about common lid take-up spindle 316 in the common lid take-up chamber 318.

It will be noted that common lid take-up spindle 316 comprises plural arms 317 that splay out radially from the centre to give it an overall ‘collapsible wheel’ form. In use, as lid-foil 320a, 320b wraps around the spindle 316, the arms 317 collapse inwardly thereby reducing the diameter of the spindle 316 itself but acting to maintain a roughly constant effective winding diameter as defined by the diameter of the spindle 316 in combination with the used lid foil 320a, 320b wrapped there around. The maintenance of this constant effective winding diameter ensures uniform indexing of each strip 301a, 301b over the entire strip length.

In use, the dispenser is primed by actuating lever 326 located on the side of the dispenser to drivably actuate the lid-take up spindle 316 to advance each blister strip 301a, 301b, thereby causing the leading pocket 304a, 304b thereof to be peeled open. To access the contents of the opened pockets 304a, 304b, the patient then breathes in through the outlet 324. This results in negative pressure being transmitted through manifold 322 to the opened leading pocket 304a, 304b of each strip 301a, 301b at the opening station 308. This in turn, results in the medicament powder contained within each of the opened pockets 304a, 304b being drawn out through the common manifold 322 to the outlet 324 and hence to the patient as an inhaled combination medicament dose. It be appreciated that, mixing of each separately delivered component of the combined medicament product happens as the powder is transported from each opened pocket 304a, 304b to the outlet 324.

Importantly, the dispenser of FIG. 3 enables different medicament types to be stored separately in each of the strips 301a, 301b but allows for the release and delivery thereof to the patient via the single outlet 324 as a combined inhaled product.

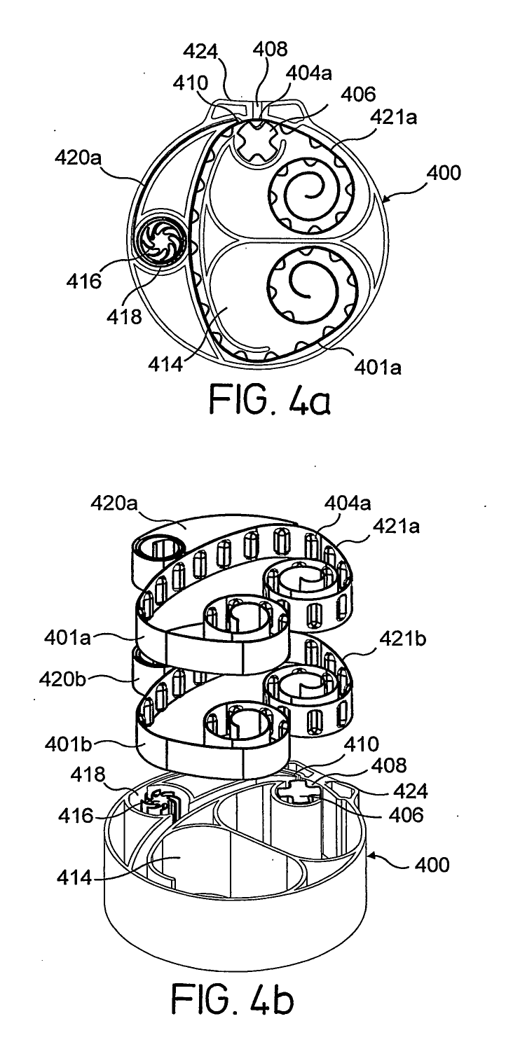

FIGS. 4a and 4b respectively illustrate sectional and perspective views of base unit 400 of a medicament dispenser according to the invention. In use, a protective cover 30 (not shown) would be provided to the base unit 400. First and second medicament-containing blister strips 401a, 401b are positioned one on top of the other (in ‘double-decker’ configuration) in the base unit 400. The first blister strip 401a contains multiple dose portions of a first active medicament component. The second blister strip 401b contains multiple dose portions of a second active medicament component. In this configuration, each blister strip 401a, 401b shares the same internal mechanism elements (e.g. drive, index, opening) of the base unit 400. Thus, each strip 401a, 401b engages shared multi-pocket index wheel 406 and successive pockets are thereby guided towards a central opening station 408. At the opening station 408, the lid foil 420a, 420b and base foil 421a, 421b parts of each strip 401a, 401b are peelably separable about beak 410. The resulting empty base foil 421a, 421b coils up in base take-up chamber 414. The used lid foil 420a, 420b feeds over beak 410 and coils about common ‘collapsible wheel’ form lid take-up spindle 416 in the common lid take-up chamber 418.

In use, the dispenser is primed by drivably actuating the lid-take up spindle 416 to advance each blister strip 401a, 401b, thereby causing the leading pocket 404a (leading pocket not visible on second strip) thereof to be peeled open. To access the contents of the opened pocket 404a the patient then breathes in through the outlet 424. This results in negative pressure being transmitted to the opened leading pockets 404a at the opening station 408. This in turn, results in the medicament powder contained within the opened pocket 404a of each strip 401a, 401b being drawn out to the outlet 424 and hence to the patient as an inhaled combination medicament dose.

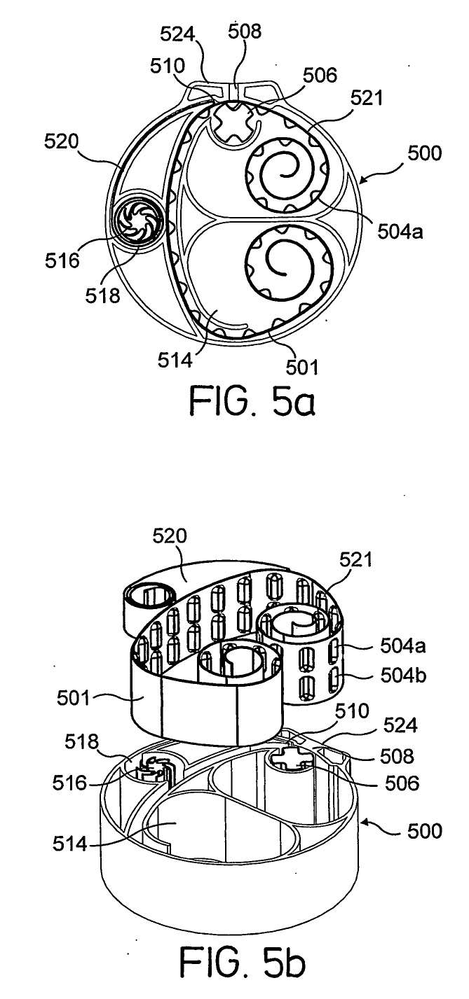

FIGS. 5a and 5b respectively illustrate sectional and perspective views of base unit 500 of a medicament dispenser that may be appreciated to be a variation of the dispenser of FIG. 4. In the dispenser of FIG. 4, the ‘double decker’ configuration of separate strips 501a, 501b of FIG. 4 is replaced by a single strip 401 comprising dual series of pockets 404a, 404b arranged in parallel fashion thereon for receipt by the base unit 400. The first series of pockets 404a contains multiple dose portions of a first active medicament component. The second series of pockets 404b contains multiple dose portions of a second active medicament component.

As with the dispenser of FIG. 4, each series of blister pockets 404a, 404b shares the same internal mechanism elements (e.g. drive, index, opening) of the base unit 400. Thus, the dual series strip 401 engages multi-pocket index wheel 406 and successive pockets of both series are thereby guided towards a central opening station 408. At the opening station 408, the lid foil 420 and base foil 421 parts of the dual series strip 401 are peelably separable about beak 410. The resulting empty base foil 421 coils up in base foil take-up chamber 414. The used lid 420 feeds over beak 410 and coils about common ‘collapsible wheel’ form lid take-up spindle 416 in the common lid take-up chamber 418.

In use, the dispenser is primed by drivably actuating the lid-take up spindle 416 to advance the dual series blister strip 401 thereby causing the leading pockets 404a, 404b of each series thereof to be peeled open. To access the contents of the opened pockets 404a, 404b the patient then breathes in through the outlet 424. This results in negative pressure being transmitted to the opened leading pockets 404a, 404b at the opening station 408. This in turn, results in the medicament powder contained within each of the opened pockets 404a, 404b being drawn out to the outlet 424 and hence to the patient as an inhaled combination medicament dose.

As shown in FIG. 5, the pockets of each series are of equivalent size and shape. It will be appreciated, that in variations, the pockets of one series may be shaped and/or sized differently from that of another series.

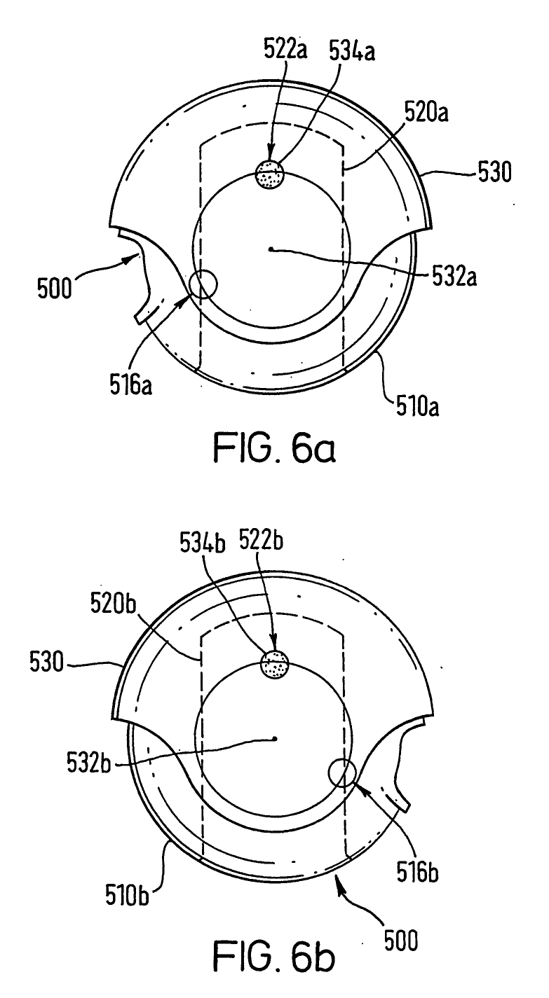

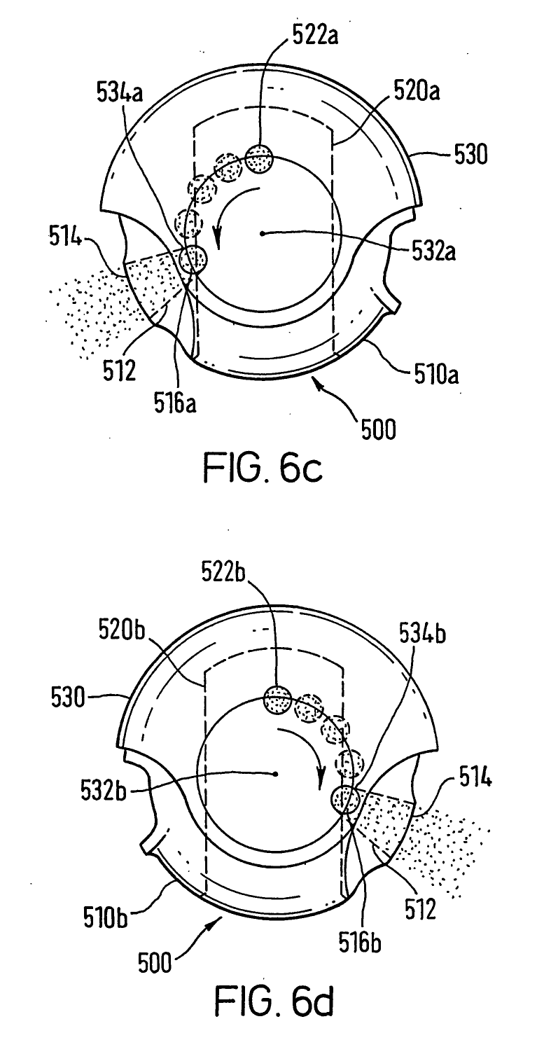

FIGS. 6a and 6b respectively show top and bottom views of a reservoir DPI dispenser herein in a non-dispensing position. FIGS. 6c and 6d respectively show top and bottom views of the dispenser in a dispensing position. The dispenser comprises a generally circular body 500 comprised of two clamshell halves 510a, 510b mating together to define a common outlet 512 defining a mouthpiece 514 (only visible in FIGS. 6c and 6d). Each clamshell half 510a, 510b is also provided with a dispensing orifice 516a, 516b which in the open position communicates with the common outlet 512 and mouthpiece 514 for dispensing of medicament therethrough. Defined by each clamshell half 510a, 510b there is a respective medicament container 520a, 520b for containment of dry powder medicament. The first medicament container 520a contains first active medicament component. The second medicament container 520b contains second active medicament component. Each container 520a, 520b is provided with a delivery orifice 522a, 522b for delivery of its dry powder medicament contents. A unitary cover 530 is also provided to the body 500 wherein the cover is pivotally mounted at pivot points 532a, 532b to the clamshell halves 510a, 510b of body 500 such that it is rotatable by 180° around the body 500. The inner part of the cover 530 is provided with both top and bottom metering recesses 534a, 534b. In the cover closed position, each metering recess 534a, 534b locates adjacent the delivery orifice 522a, 522b of its respective medicament container 520a, 520b such that powder may enter each metering recess 534a, 534b therefrom. In the open position, each metering recess 534a, 534b locates adjacent its respective dispensing orifice 516a, 516b such that powder may pass therethrough to the common outlet 512 and mouthpiece 514.

It may be appreciated that the device is operable by a single-handed operation in which the cover 530 is held in the cupped fingers (not shown) of a user and the body 500 rotated through 180° by a thumb action of the user to bring the device from the open to closed position and vice versa. It may also be appreciated that the 180° rotation of the cover 530 acts such as to either expose or cover the mouthpiece 514 and to move each metering recess 534a, 534b from a loading position to a dispensing position.

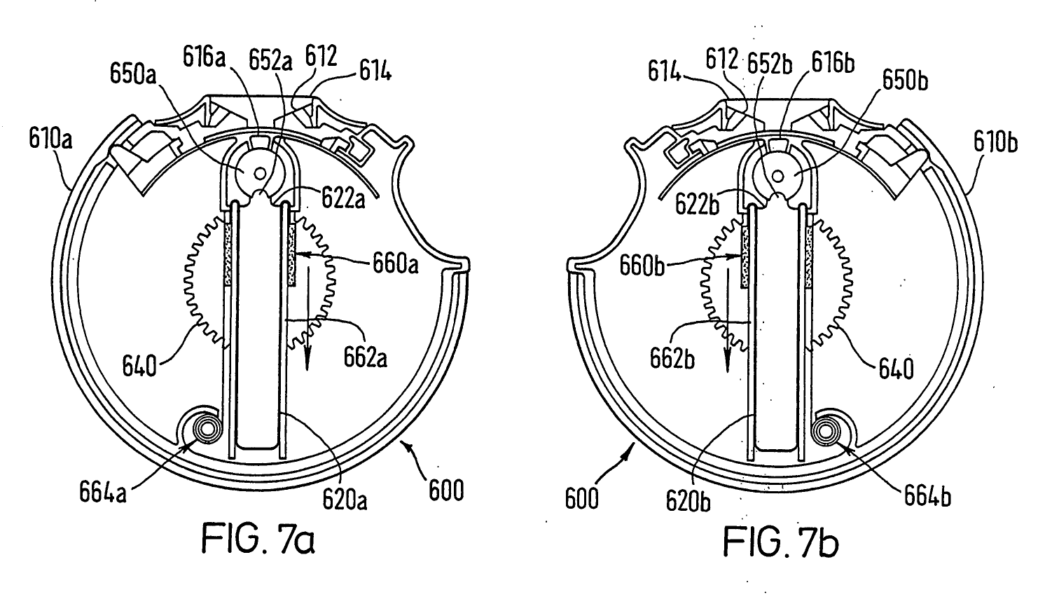

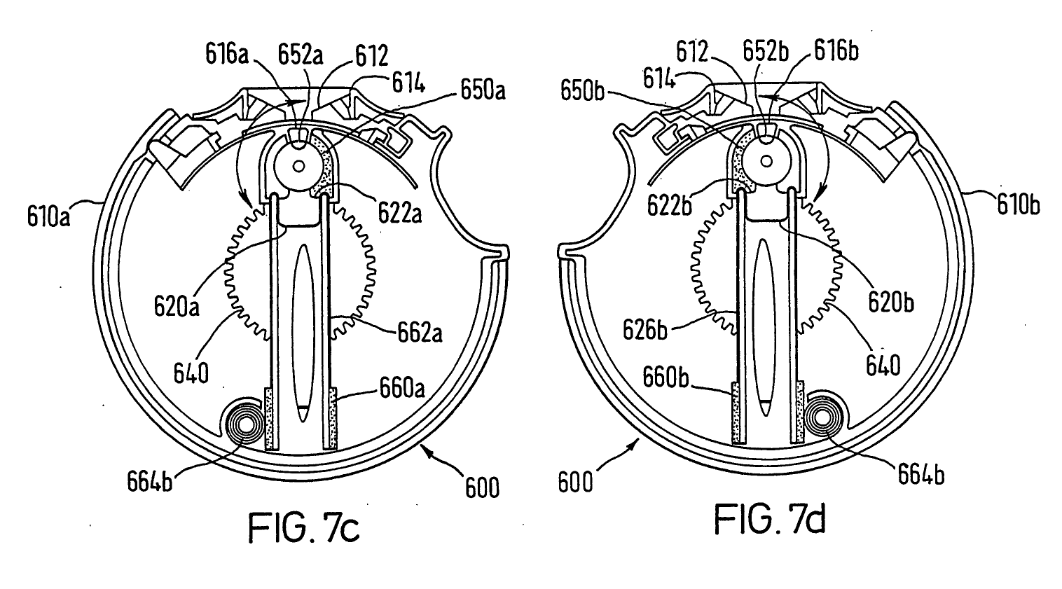

FIGS. 7a and 7b respectively show top and bottom views of another reservoir DPI dispenser herein in a non-dispensing position. FIGS. 7c and 7d respectively show top and bottom views of the dispenser in a dispensing position. The dispenser comprises a generally circular body 600 comprised of two clamshell halves 610a, 610b mating together to define a common outlet 612 defining a mouthpiece 614. Each clamshell half 610a, 610b is also provided with a dispensing orifice 616a, 616b that in the open position communicates with the common outlet 612 and mouthpiece 614 for dispensing of medicament therethrough. Provided within each clamshell half 610a, 610b there is a respective collapsible tube container 620a, 620b for containment of dry powder medicament. The first medicament container 620a contains first active medicament component. The second medicament container 620b contains second active medicament component. Each container 620a, 620b is provided with a delivery orifice 622a, 622b for delivery of powder. A cover (not visible) is provided to the body 600 wherein the cover is pivotally mounted such that it is rotatable by 180° around the body 600. The cover is co-axially mounted and rotationally coupled to common drive wheel 140 such that rotation of the cover results in rotation of the drive wheel 640. The common drive wheel 640 engages first and second metering wheels 650a, 650b each of which is provided with a metering recess 652a, 652b. In the cover-closed position, each metering recess 652a, 652b locates adjacent the delivery orifice 622a, 622b of its respective medicament cartridge 620a, 620b such that powder may enter each metering recess 652a, 652b therefrom. In the open position, each metering recess 652a, 652b locates adjacent its respective dispensing orifice 616a, 616b such that powder may pass therethrough to the common outlet 612 and mouthpiece 614.

It may be seen that each powder container 620a, 620b is also provided with a system for ensuring constant delivery of powder to its delivery orifice 622a, 622b. The system comprises a collar 660a, 660b movable along a track 662a, 662b located on either side of the tubular container 620a, 620b. Pulling force is applied to the collar 660a, 660b by constant force spring 664a, 664b. As each collar 660a, 660b is pulled along its track 662a, 662b by the action of its spring 664a, 664b the tube 620a, 620b is squeezed and powder is urged towards its delivery orifice 622a, 622b.

It may be appreciated that the dispenser of FIGS. 7a to 7d is operable by a single-handed operation in which the cover (not shown) is held in the cupped fingers (not shown) of a user and the body 600 rotated through 180° by a thumb action of the user to bring the device from the open to closed position and vice versa. It may also be appreciated that the 180° rotation of the cover acts such as to both expose or cover the mouthpiece 614 and move each metering recess 652a, 652b from a loading position to a dispensing position.

In a variation of the dispenser of FIGS. 7a to 7d, the common drive wheel 640 is replaced by first and second independent drive wheels, each of which drives respective first and second metering wheels 650a, 650b. In this variation, the movement of each independent drive wheel is independently and releasably couplable to the movement of the cover. In one mode of use, only one independent drive wheel is coupled to the cover such that cover movement results only in movement of that drive wheel and its corresponding metering wheel 650a such that powder is metered from only one medicament container 620a. In another mode of use, both independent drive wheels are coupled to the cover such that cover movement results in movement both drive wheels and corresponding metering wheels 650a, 650b such that powder is metered from both medicament containers 620a, 620b. In this variation, the dispenser may thus be arranged to deliver only one medicament powder or two medicament powders as a combination product.

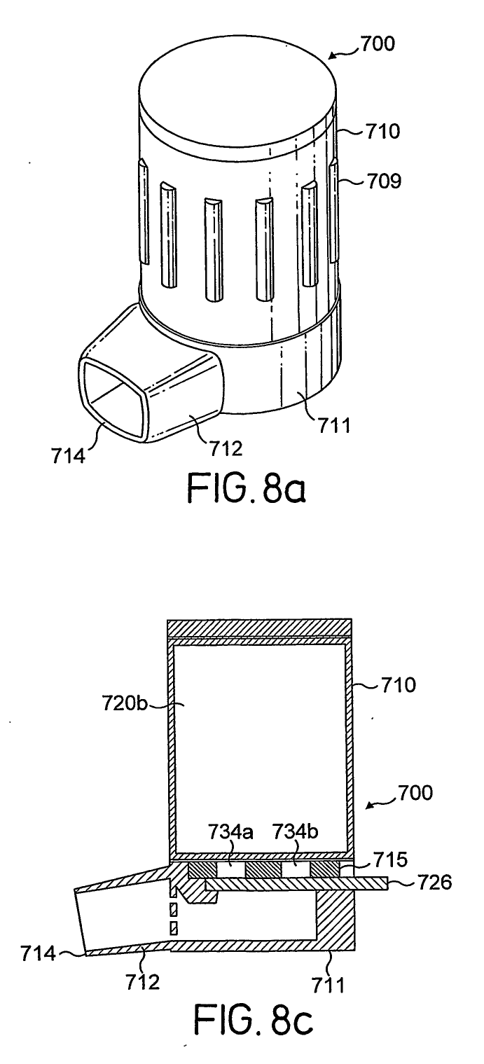

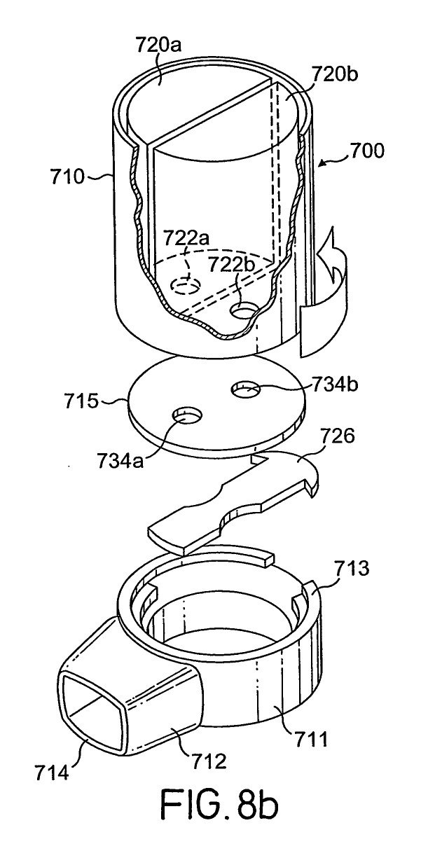

FIGS. 8a to 8c illustrate a third reservoir dispenser herein, as shown respectively in perspective, exploded and sectional side views. The dispenser comprises a generally L-shaped body 700 comprised of upper column-shaped housing 710 rotationally mounted to base 711. The base 711 is shaped to define a common outlet 712 in the form of a mouthpiece 714. The column-shaped housing 710 has grips 709 for ease of patient grip, and is provided with two medicament containers 720a, 720b (both visible in FIG. 8b only) of semi-circular cross-section, each for containment of dry powder medicament. The first medicament container 720a contains first active medicament component. The second medicament container 720b contains second active medicament component. Each container 720a, 720b is itself provided with circular delivery orifice 722a, 722b for delivery of its dry powder medicament contents. Locating within the upper rim 713 of the base 711 and fixedly mounted with respect thereto, there is provided circular plate 715. The plate has two circular metering orifices 734a, 734b, each sized and shaped to register with the circular delivery orifices 722a, 722b of the respective containers 720a, 720b, in a metering position. Dispensing lever 726 locates beneath the plate 715 and is mounted for rotation with respect to the base 711. The Lever is rotationally movable from a non-dispensing position in which it acts to close off communication between the metering orifices 734a, 734b of the plate 715 to a dispensing position in which the metering orifices 734a, 734b communicate with the common outlet 712 and mouthpiece 714 of the base 711 for dispensing of medicament therethrough.

Usage of the dispenser of FIGS. 8a to 8c involves two distinct actions, namely metering and dispensing. In the metering action, the column 710 is rotated with respect to the base 711 until the circular delivery orifices 722a, 722b of the respective containers 720a, 720b are brought into registration with the circular metering orifices 734a, 734b of the plate 715. A metered quantity of the medicament powder contents of each container 720a, 720b is thereby delivered under gravity to each metering orifice 734a, 734b. The column 711 is then rotated in a reverse sense to bring the respective orifices 722a, 722b and 734a, 734b out of registration with each other but leaving a metered quantity of medicament powder in each metering orifice 734a, 734b. It will be appreciated that in the metering stage, the lever 726 is in the non-dispensing (i.e. closed off) position with respect to the plate 715.

In the dispensing action, the lever 726 is now rotated from the non-dispensing position in which it acts to close off communication between the metering orifices 734a, 734b of the plate 715 to the dispensing position in which the volume of medicament powder contained within each metering orifice 734a, 734b is released to the base for dispensing to an inhaling patient through the common outlet 712 and mouthpiece 714.

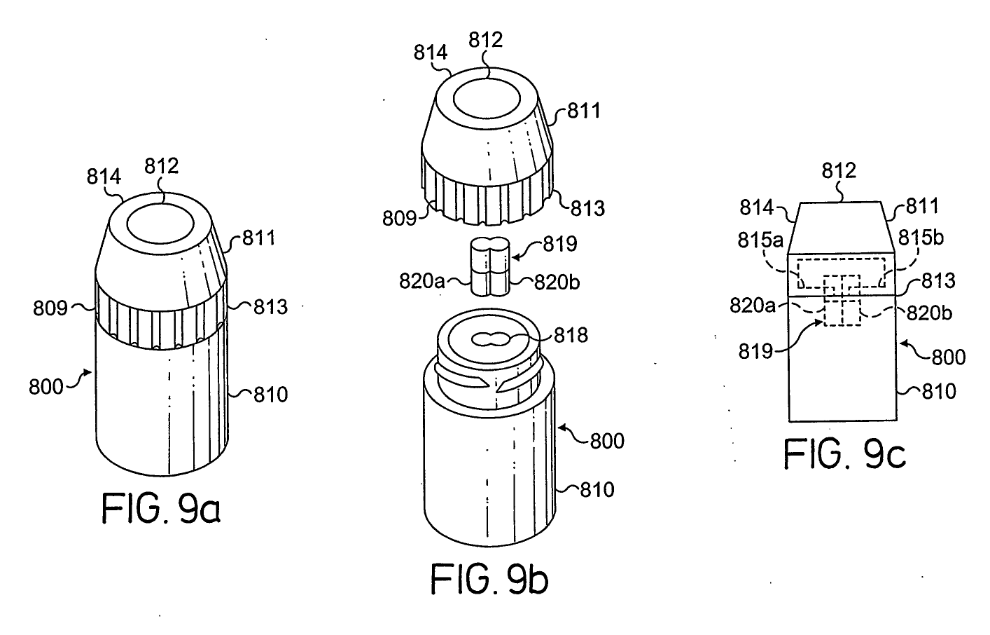

FIGS. 9a to 9c illustrate a first DPI capsule dispenser herein, as shown respectively in perspective, exploded and sectional side views. The dispenser comprises a generally cylindrical body 800 comprised of column housing 810 rotationally mounted to dispensing head 811. The head 811 is shaped to define a common outlet 812 in the form of a mouthpiece 814 and has grips 809 for ease of patient grip thereof. The housing 810 is provided with dual-lobed cavity 818 shaped for receipt of dual-lobed capsule body 819, which defines two separate medicament containers 820a, 820b, each for containment of dry powder medicament. In essence, the dual-lobed capsule 819 acts as a simple type of ‘refill cassette’ comprising dual medicament containers 820a, 820b. The first medicament container 820a contains a unit dose portion of a first active medicament component. The second medicament container 820b contains a unit dose portion of a second active medicament component. Located at the mating rim 813 of the head 811 and fixedly mounted with respect thereto, there are provided two jutting inner edge features 815a, 815b at 180° rotational spacing relative to each other. The head 811 is rotatable relative to the housing from a first position in which the jutting inner edges 815a, 815b thereof are distant from the dual-lobed capsule 819 to a second position in which the edges 815a, 815b destructively interact with the capsule to sever each respective container part 820a, 820b thereof. Once the capsule is severed, the medicament powder held within each container 820a, 820b is made available for inhalation by a patient through the common outlet 812 and mouthpiece 814.

Usage of the dispenser of FIGS. 9a to 9c involves accessing of the capsules and then dispensing the contents thereof to a patient. In the capsule accessing action, the housing 810 is rotated with respect to the head 811 until the jutting edges 815a, 815b sever the dual-lobed capsule 819 to enable access to contents of the medicament containers 820a, 820b. The patient then inhales through the mouthpiece 814 to aerosolise the powder contained in each container 820a, 820b of the capsule 819 for delivery as a combination product through the common outlet 812 and mouthpiece 814.

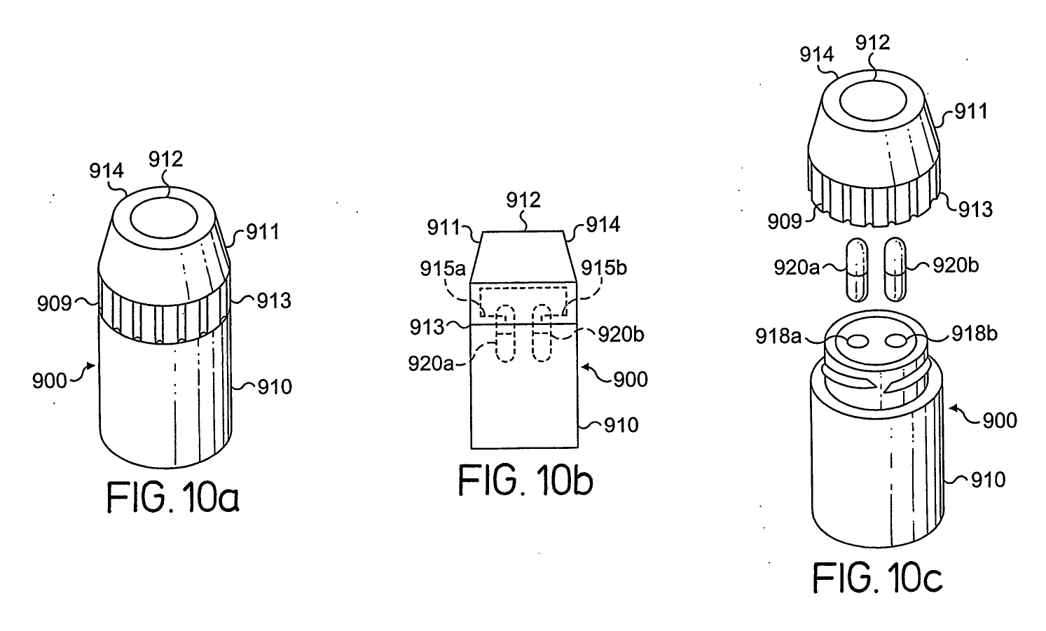

FIGS. 10a to 10c illustrate a second DPI capsule dispenser herein, as shown respectively in perspective, exploded and sectional side views. It will be appreciated that this dispenser is a dual capsule variation of the first DPI dispenser shown in FIGS. 9a to 9c. The dispenser comprises a generally cylindrical body 900 comprised of column housing 910 rotationally mounted to dispensing head 911. The head 911 is shaped to define a common outlet 912 in the form of a mouthpiece 914 and has grips 909 for ease of patient grip thereof. The housing 910 is provided with dual cavities 918a, 918b, each shaped for receipt of a medicament container 920a, 920b in the form of a capsule for containing dry powder medicament. The first medicament container 920a contains a unit dose portion of a first active medicament component. The second medicament container 920b contains a unit dose portion of a second active medicament component. Located at the mating rim 913 of the head 911 and fixedly mounted with respect thereto, there are provided two jutting inner edge features 915a, 915b at 180° rotational spacing relative to each other. The head 911 is rotatable relative to the housing from a first position in which the jutting inner edges 915a, 915b thereof are distant from respective capsules 920a, 920b to a second position in which each edge 915a, 915b destructively interacts with a capsule 920a, 920b to sever it open Once each capsule 920a, 920b is severed, the medicament powder held within it is made available for inhalation by a patient through the common outlet 912 and mouthpiece 914.

Usage of the dispenser of FIGS. 10a to 10c involves accessing of the capsules and then dispensing the contents thereof to a patient. In the capsule accessing action, the housing 910 is rotated with respect to the head 911 until the jutting edges 915a, 915b sever the capsule 920a, 920b to enable access to the dry powder medicament contents thereof. The patient then inhales through the mouthpiece 914 to aerosolise the powder contained in each capsule 920a, 920b for delivery as a combination product through the common outlet 912 and mouthpiece 914.

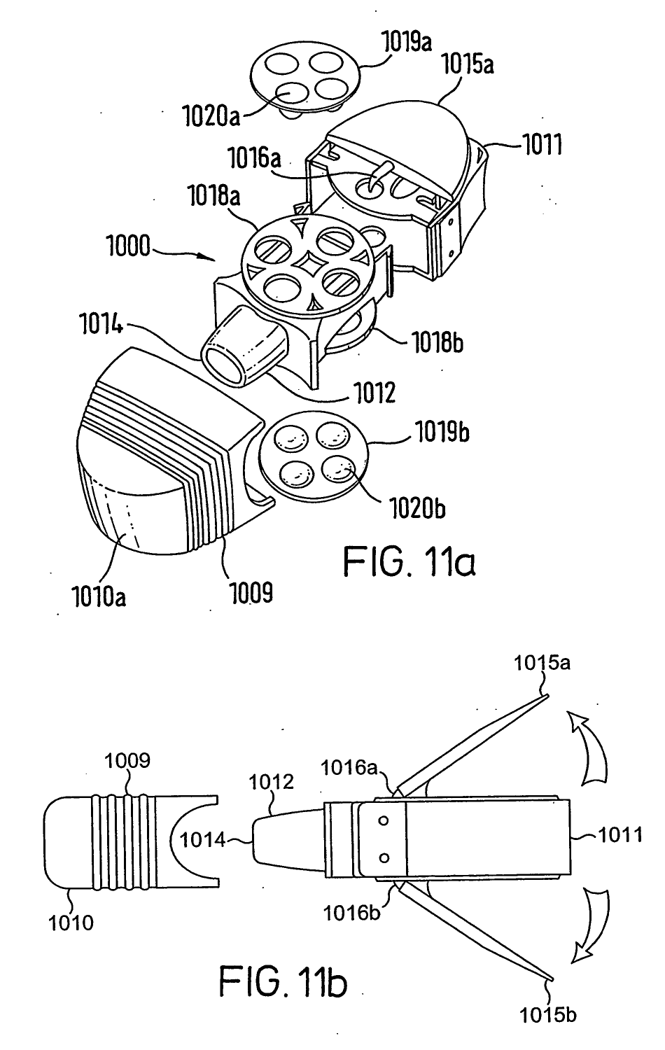

FIGS. 11a and 11b show a fourth MDPI dispenser herein respectively in exploded perspective and side views. The dispenser comprises a central body 1000 comprising upper and lower, rotationally mounted circular carriages 1018a, 1018b, each shaped for receipt of a circular medicament carrier disk 1019a, 1019b. Each disk 1019a, 1019b has provided thereto four, evenly spaced blisters 1020a, 1020b for containing medicament powder. The first blister pack 1019a contains multiple dose portions of a first active medicament component. The second blister pack 1019b contains multiple dose portions of a second active medicament component. Variations involving, for example, six and eight blisters disks 1019a, 1019b are also envisaged. The body 1000 is also provided with a common outlet 1012 defining a mouthpiece 1014.

The body 1000 of the dispenser is housed within an elongate housing comprised of two mating halves 1010, 1011. One half 1010 acts as a cover for the mouthpiece 1014 and is provided with finger grips 1009 for ease of its removal by a patient. The second half 1011 is provided with upper and lower, hingedly mounted wings 1015a, 1015b. The tip of each wing 1015a, 1015b is provided with a piercing element 1016a, 1016b for enabling piercable access to a blister 1020a, 1020b of an associated disk 1019a, 1019b, thereby enabling release of dry powder medicament therefrom. Once so released, the dry powder is made available for inhalation by a patient via the common outlet 1012 and mouthpiece 1014.

In use, each carriage 1018a, 1018b is first rotated to bring an unopened blister 1020a, 1020b of each disk 1019a, 1019b to a position where it may be piercably accessed. The upper and lower wings 1015a, 1015b are then squeezed towards each other to bring the piercing element 1016a, 1016b of each into piercing contact with the blister 1020a, 1020b thereby piercing it open. The patient then inhales through the mouthpiece 1014 to aerosolise the dry powder medicament contents of each opened blister 1020a, 1020b and draw such contents via common outlet 1012 and mouthpiece 1014 for inhalation as a combination product.

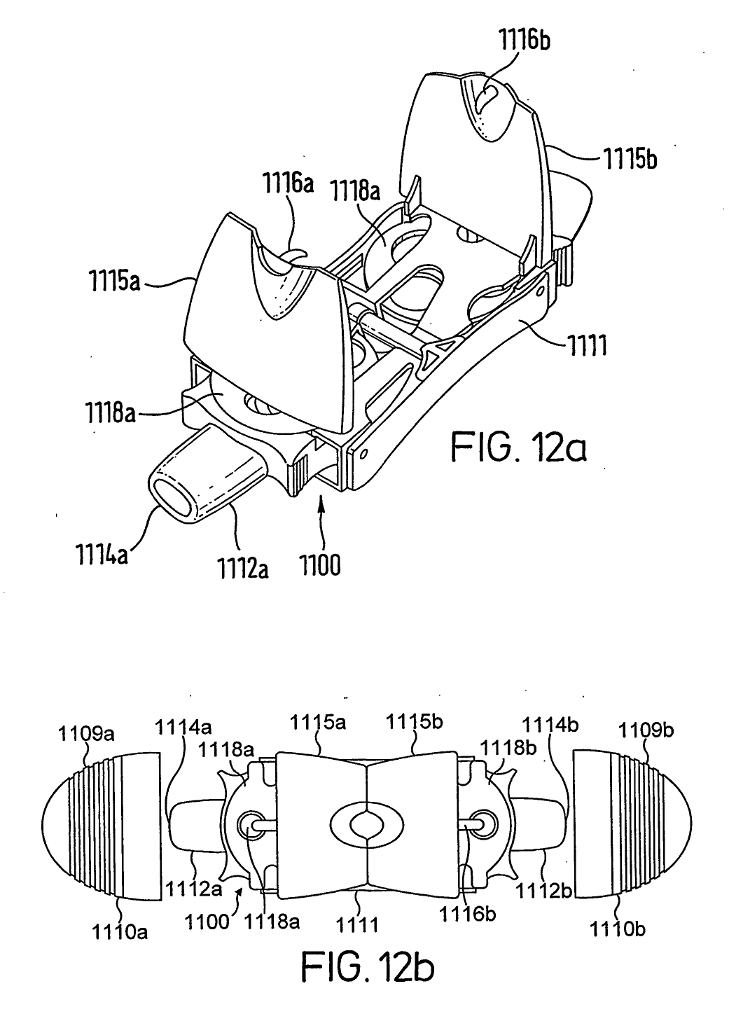

FIGS. 12a and 12b show a fifth MDPI dispenser herein respectively in perspective and exploded top views. The dispenser comprises a central body 1100 comprising first and second, rotationally mounted circular carriages 1118a, 1118b, each shaped for receipt of a circular medicament carrier disk (not shown). As in FIGS. 11a and 11b, each disk will have provided thereto from four to eight evenly spaced blisters for containing medicament powder. The first blister pack 1119a contains multiple dose portions of a first active medicament component. The second blister pack 1119b contains multiple dose portions of a second active medicament component. The body 1100 is also provided with first and second outlets 1112a, 1112b, each defining a mouthpiece 1114a, 1114b.

The body 1100 of the dispenser is housed within an elongate housing comprised of central trunk 1111 and mating end-covers 1110a, 1110b provided thereto. It will be seen that each end-cover 1110a, 1110b acts as a cover for a respective mouthpiece 1114a, 1114b and is provided with finger grips 1109a, 1109b for ease of removal by a patient. The central trunk 1111 is provided with first and second, hingedly mounted wings 1115a, 1115b. The tip of each wing 1115a, 11115b is provided with a piercing element 1116a, 1116b for piercing access to a blister of an associated disk, thereby enabling release of dry powder medicament therefrom. Once so released, dry powder is made available for inhalation by a patient via each outlet 1112a, 1112b and mouthpiece 1114a, 1114b.

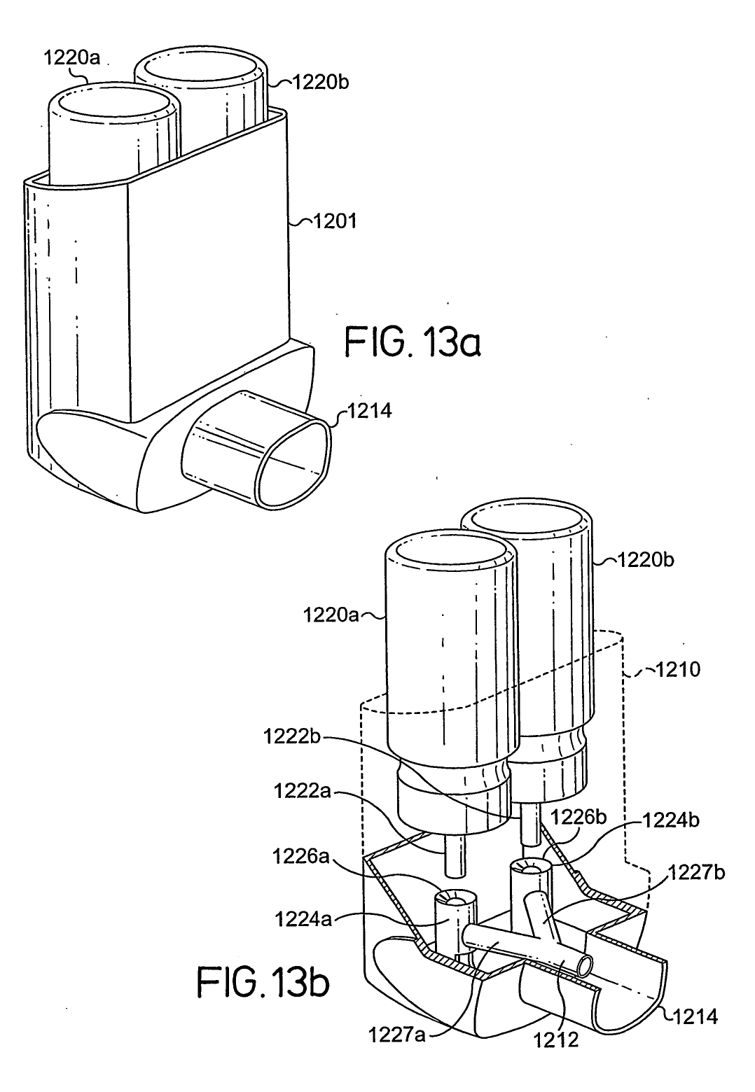

In use, each carriage 1118a, 1118b is first rotated to bring an unopened blister of each disk to a position where it may be piercably accessed. The first and second wings 1115a, 1115b are then squeezed towards each other to bring the piercing element 1116a, 1116b of each into piercing contact with the blister thereby piercing it open. The patient then inhales through the first mouthpiece 1114a to aerosolise the dry powder medicament contents of the opened blister of the first disk and draw such contents via first outlet 1112a and mouthpiece 1114a for inhalation. Subsequently, the patient inhales through the second mouthpiece 1114b to aerosolise the dry powder medicament contents of the opened blister of the second disk and draw such contents via first outlet 1112b and mouthpiece 1114b for inhalation. It will thus, be appreciated that the dispenser of FIGS. 12a and 12b is suitable for the sequential dispensing of the separate components of a combination product, in contrast to the related dispenser of FIGS. 11a and 11b which is suitable for combined dispensing. FIG. 13a shows a perspective view of a dual MDI dispenser herein and FIG. 13b shows the dispenser of FIG. 13a in part cut-away view. The dual MDI dispenser comprises a generally L-shaped tubular housing 1201 shaped for receipt of two aerosol containers 1220a, 1220b. The first aerosol container 1220a contains first active medicament component in aerosol formulation form. The second aerosol container 1220b contains second active medicament component in aerosol formulation form. The housing is open at one end (which will hereinafter be considered to be the top of the device for convenience of description) and is closed at the other. A common mouthpiece 1214 leads laterally from the closed end of the housing 1201. In variations, the mouthpiece 1214 may if desired, be designed as a nozzle for insertion into the patient's nostril.

Each aerosol container 1220a, 1220b has an outlet valve stem 1222a, 1222b at one end. Each valve 1222a, 1222b can be depressed to release a metered dose from its respective aerosol container 1220a, 1220b. Each aerosol container 1220a, 1220b locates in the housing 1201 such that one end protrudes from its open top. Valve support 1224a, 1224b are provided at the lower end of the housing 1201 and are provided with respective passages 1226a, 1226b in which the valve stem 1222a, 1222b of each respective aerosol container 1220a, 1220b can be located and supported. A second passage 1227a, 1227b leads from each support 1224a, 1224b and is directed towards common outlet passage 1212.

In use, the protruding portion of each aerosol container 1220a, 1220b is depressed to move that container 1220a, 1220b relative to its valve stem 1222a, 1222a to open the valve and discharge an aerosol form dose of medicament through passages 1227a, 1227b to common outlet passage 1212 and thence to the mouthpiece 1214 from which it can be inhaled by a patient. A measurement amount (e.g. a part-combination dose) will be released from each aerosol container 1220a, 1220b each time it is fully depressed.

It will be appreciated that the required depression of each aerosol container 1220a, 1220b is achievable by a dual fingered patient action whilst the base of the housing 1201 is held in a patient's cupped hand. In variations, the movement of the containers 1220a, 1220b may be coupled (e.g. through use of a coupling element) thereby ensuring that both containers 1220a, 1220b are fired in tandem.

In a first particular set of examples, the first medicament active component of the each dispenser shown in FIGS. 2a to 13b is salmeterol xinafoate and the second medicament active component is fluticasone propionate.

In a second particular set of examples, the first medicament active component of the each dispenser shown in FIGS. 2a to 13b is formoterol fumarate and the second medicament active component is fluticasone propionate.

In a third particular set of examples, the first medicament active component of the each dispenser shown in FIGS. 2a to 13b is formoterol fumarate and the second medicament active component is beclomethasone dipropionate.

It may be appreciated that any of the parts of the dispenser (or refill/cassette) that contact the medicament suspension may be coated with materials such as fluoropolymer materials (e.g. PTFE or FEP) which reduce the tendency of medicament to adhere thereto. Any movable parts may also have coatings applied thereto which enhance their desired movement characteristics. Frictional coatings may therefore be applied to enhance frictional contact and lubricants (e.g. silicone oil) used to reduce frictional contact as necessary.

The medicament dispenser of the invention is suitable for dispensing medicament combinations, particularly for the treatment of respiratory disorders such as asthma and chronic obstructive pulmonary disease (COPD), bronchitis and chest infections.

The first medicament active component is selected from the group consisting of salmeterol, formoterol and any salts or solvates thereof. The second medicament active component is selected from the group consisting of beclomethasone ester, fluticasone ester and any salts or solvates thereof.

Other medicaments may also be employed in addition to the first and second active medicaments. Appropriate other medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g. flunisolide, budesonide, rofleponide, mometasone e.g. as the furoate ester), ciclesonide, triamcinolone (e.g. as the acetonide) or 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), ephedrine, adrenaline, fenoterol (e.g. as hydrobromide), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy) acetyl]amino}pentanoyl)amino] propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

The first or second active medicaments may be provided in pure drug form for delivery by one of the suitable dispensers described herein. More commonly however, the medicaments will be formulated (e.g. as a dry powder or aerosol type formulation).

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as pure drug, but more appropriately, it is preferred that medicaments are delivered together with excipients (carriers) which are suitable for inhalation. Suitable excipients include organic excipients such as polysaccharides (i.e. starch, cellulose and the like), lactose, glucose, mannitol, amino acids, and maltodextrins, and inorganic excipients such as calcium carbonate or sodium chloride. Lactose is a preferred excipient.

Particles of the powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

The excipient may be included with the medicament via well-known methods, such as by admixing, co-precipitating and the like. Blends of excipients and drugs are typically formulated to allow the precise metering and dispersion of the blend into doses. A standard blend, for example, contains 13000 micrograms lactose mixed with 50 micrograms drug, yielding an excipient to drug ratio of 260:1. Dosage blends with excipient to drug ratios of from 100:1 to 1:1 may be used. At very low ratios of excipient to drug, however, the drug dose reproducibility may become more variable.

Either of the first and second active medicaments may be provided in dry powder form for delivery by an RDPI, MDPI or UDPI type dispenser.

Either of the first and second active medicaments may be provided in the form of an aerosol formulation for delivery by an MDI type dispenser. Suitably, the aerosol formulation comprises the active medicament and a fluorocarbon or hydrogen-containing chlorofluorocarbon propellant.

Suitable propellants include, for example, C₁₋₄hydrogen-containing chlorofluorocarbons such as CH₂ClF, CClF₂CHClF, CF₃CHClF, CHF₂CClF₂, CHClFCHF₂, CF₃CH₂Cl and CClF₂CH₃; C₁₋₄hydrogen-containing fluorocarbons such as CHF₂CHF₂, CF₃CH₂F, CHF₂CH₃ and CF₃CHFCF₃; and perfluorocarbons such as CF₃CF₃ and CF₃CF₂CF₃.

Where mixtures of the fluorocarbons or hydrogen-containing chlorofluorocarbons are employed they may be mixtures of the above identified compounds or mixtures, preferably binary mixtures, with other fluorocarbons or hydrogen-containing chloro-fluorocarbons for example CHClF₂, CH₂F₂ and CF₃CH₃. Preferably a single fluorocarbon or hydrogen-containing chlorofluorocarbon is employed as the propellant. Particularly preferred as propellants are C₁₋₄hydrogen-containing fluorocarbons such as 1,1,1,2-tetrafluoroethane (CF₃CH₂F) and 1,1,1,2,3,3,3-heptafluoro-n-propane (CF₃CHFCF₃) or mixtures thereof.

Preferably, the aerosol formulations are substantially free of chlorofluorocarbons such as CCl₃F, CCl₂F₂ and CF₃CCl₃.

The propellant may additionally contain a volatile adjuvant such as a saturated hydrocarbon for example propane, n-butane, isobutane, pentane and isopentane or a dialkyl ether for example dimethyl ether. In general, up to 50% w/w of the propellant may comprise a volatile hydrocarbon, for example 1 to 30% w/w. However, formulations, which are free or substantially free of volatile adjuvants are preferred. In certain cases, it may be desirable to include appropriate amounts of water, which can be advantageous in modifying the dielectric properties of the propellant.

Preferably, the aerosol formulation contains propellants (including propellant mixtures) which are more hygroscopic than P11, P114 and/or P12 such as HFA-134a and HFA-227.

A polar co-solvent such as C₂₋₆ aliphatic alcohols and polyols e.g. ethanol, isopropanol and propylene glycol, preferably ethanol, may be included in the aerosol formulation in the desired amount to improve the dispersion of the formulation, either as the only excipient or in addition to other excipients such as surfactants. Suitably, the aerosol formulation may contain 0.01 to 30% w/w based on the propellant of a polar co-solvent e.g. ethanol, preferably 0.1 to 20% w/w e.g. about 0.1 to 15% w/w. In aspects herein, the solvent is added in sufficient quantities to solubilise part or all of the active medicament component, such formulations being commonly referred to as solution formulations.

A surfactant may also be employed in the aerosol formulation. Examples of conventional surfactants are disclosed in EP-A-372,777. The amount of surfactant employed is desirable in the range 0.0001% to 50% weight to weight ratio relative to the medicament, in particular, 0.05 to 5% weight to weight ratio.

The final aerosol formulation desirably contains 0.005-10% w/w, preferably 0.005 to 5% w/w, especially 0.01 to 1.0% w/w, of medicament relative to the total weight of the formulation.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. A medicament dispenser for use in the delivery of a combination medicament product, the dispenser comprising
a first medicament container for containing a first medicament active component;
a first release means for releasing a dose portion of said first medicament active component from said first medicament container;
a second medicament container for containing a second medicament active component; and
a second release means for releasing a dose portion of said second medicament active component from said second medicament container,
wherein the first medicament active component is kept separate from the second medicament active component until the point of release thereof for delivery in combination, and wherein the first medicament active component is selected from the group consisting of salmeterol, formoterol and any salts or solvates thereof and the second medicament active component is selected from the group consisting of beclomethasone ester, fluticasone ester and any salts or solvates thereof.

2. A medicament dispenser according to claim 1, wherein the first medicament active component is selected from the group consisting of salmeterol xinafoate and formoterol fumarate.

3. A medicament dispenser according to claim 1, wherein the second medicament active component is selected from the group consisting of beclomethasone dipropionate and a fluticasone propionate.

4. A medicament dispenser according to claim 1, wherein the first medicament active component is salmeterol xinafoate and the second medicament active component is fluticasone propionate.

5. A medicament dispenser according to claim 1, wherein the first medicament active component is formoterol fumarate and the second medicament active component is fluticasone propionate.

6. A medicament dispenser according to claim 1, wherein the first medicament active component is formoterol fumarate and the second medicament active component is beclomethasone dipropionate.

7. A medicament dispenser according to claim 1, wherein each of the first and second medicament container are in the form of a unit dose container pack.

8. A medicament dispenser according to claim 7, wherein said unit dose container pack has capsule form.

9. A medicament dispenser according to claim 7, wherein said unit dose container pack has blister pack form.

10. A medicament dispenser according to claim 7, wherein said unit dose container pack has the form of a carrier onto which medicament has been applied by a process selected from the group consisting of printing, painting and vacuum occlusion.

11. A medicament dispenser according to claim 1, wherein each of the first and second medicament container are in the form of a reservoir form container pack for containing multiple dose portions of each active medicament component.

12. A medicament dispenser according to claim 11, including metering means for metering a dose portion of each medicament active from a reservoir container pack to a delivery position.

13. A medicament dispenser according to claim 12, wherein each metering means comprises a metering cup movable from a first position where said cup is filled with medicament from a reservoir container pack to a second position where the metered medicament dose portion in the cup is made available to the patient for inhalation.

14. A medicament dispenser according to claim 1, wherein each of the first and second medicament containers comprises a multi-dose container pack containing multiple, defined dose portions of active medicament product.

15. A medicament dispenser according to claim 14, wherein said multi-dose container pack has multi-capsule form.

16. A medicament dispenser according to claim 14, wherein said multi-dose container pack has blister pack form.

17. A medicament dispenser according to claim 16, wherein said multi-dose blister pack comprises plural blisters arranged in generally circular fashion on a disc-form blister pack.

18. A medicament dispenser according to claim 16, wherein said multi-dose blister pack comprises plural blisters arranged in series fashion on an elongate form carrier.

19. A medicament dispenser according to claim 14, wherein said multi-dose container pack has the form of a carrier onto which plural medicament dose portions have been applied by a process selected from the group consisting of printing, painting and vacuum occlusion.

20. A medicament dispenser according to claim 7, wherein each of the first and second medicament active component are in pure drug form.

21. A medicament dispenser according to claim 7, wherein each of the first and second medicament active component are in dry powder form.

22. A medicament dispenser according to claim 21, wherein each dry powder form medicament active component is formulated with an excipient selected from the group consisting of a polysaccharide, lactose, glucose, mannitol, an amino acid, a maltodextrin, calcium carbonate, sodium chloride and any mixtures thereof.

23. A medicament dispenser according to claim 1, wherein each of the first and second medicament container are in the form of an aerosol container having a metering valve.

24. A medicament dispenser according to claim 23, wherein each said metering valve has a metering volume of from 10 to 100 μl.

25. A medicament dispenser according to claim 23, wherein each medicament active component is formulated as an aerosol formulation comprising a fluorocarbon or hydrogen-containing chlorofluorocarbon propellant.

26. A medicament dispenser according to claim 25, wherein each said aerosol formulation is substantially free of chlorofluorocarbons such as CCl₃F, CCl₂F₂ and CF₃CCl₃.

27. A medicament dispenser according to claim 25, wherein said propellant is selected from the group consisting of HFA-134a, HFA-227 and any mixtures thereof.

28. A medicament dispenser according to claim 25, wherein each said aerosol formulation additionally comprises a polar co-solvent.

29. A medicament dispenser according to claim 28, wherein said polar co-solvent is ethanol.

30. A medicament dispenser according to claim 25, wherein each said aerosol formulation additionally comprises a surfactant.

31. A medicament dispenser according to claim 1, wherein said first and second release means are coupled.

32. A medicament dispenser according to claim 1, wherein the first and second medicament containers are sized and shaped to deliver equivalent dose portions.

33. A medicament dispenser according to claim 1, wherein the first and second medicament containers are sized and shaped to deliver non-equivalent dose portions.

34. A medicament dispenser according to claim 1, in reloadable form.

35. A medicament dispenser according to claim 34, wherein the first and second medicament containers are comprised as a single refill cassette.

36. A medicament dispenser according to claim 34, wherein the first and second medicament containers are comprised as separate refills.

37. A method of dispensing a combination medicament product comprising:
(a) providing a medicament dispenser according to claim 1, and;
(b) actuating said first release means and said second release means, thereby simultaneously releasing a dose portion of said first medicament active component from said first medicament container and a dose portion of said second medicament active component from said second medicament container.
